(19) United States
(12) Patent Application Publication
(10) Pub. No.: US 2007/0161648 A1
(43) Pub. Date: Jul. 12, 2007

(54) Substituted dihydro-isoindolones useful in treating kinase disorders

(76) Inventors: Terry V. Hughes, Blue Bell, PA (US); Stuart L. Emanuel, Doylestown, PA (US); Catherine A. Rugg, Lebanon, NJ (US); Peter J. Connolly, New Providence, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/549,176

(22) Filed: Oct. 13, 2006

Related U.S. Application Data
(60) Provisional application No. 60/727,155, filed on Oct. 14, 2005.

(51) Int. Cl. A61K 31/496; A61K 31/454; A61K 31/404; C07D 403/02

(52) U.S. Cl. 514/254.06; 514/321; 514/414; 544/373; 546/200; 548/454

(57) ABSTRACT

The present invention is directed to novel substituted dihydro-isoindolone compounds of formula (I): 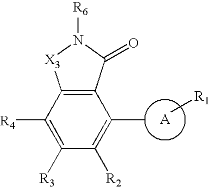
and forms thereof, wherein Ring A, X₃, R₁, R₂, R₃, R₄ and R₆ are as herein defined, and their synthesis and use as protein kinase inhibitors and interactions thereof.

### CROSS-REFERENCE TO RELATED APPLICATIONS

This present application claims benefit of U.S. Provisional Patent Application Ser. No. 60/727,155, filed Oct. 14, 2005, which is incorporated herein by reference in its entirety and for all purposes.

### FIELD OF THE INVENTION

The present invention relates to a series of substituted dihydro-isoindolone compounds, pharmaceutical compositions and methods for use thereof. In particular, the substituted dihydro-isoindolone compounds of the present invention are protein kinase inhibitors useful in treating or ameliorating a kinase mediated, angiogenesis-mediated or hyperproliferative disorder.

### BACKGROUND OF THE INVENTION

In general, protein kinases are the largest set of structurally related phosphoryl transferases, having highly conserved structures and catalytic functions. The protein kinases are categorized into families by the substrates they phosphorylate (e.g., protein-tyrosine, protein-serine/threonine, histidine and the like) and are responsible for the control of a wide variety of cellular signal transduction processes.

Examples of protein-tyrosine kinases include, but are not limited to, Irk, IGFR-1, Zap-70, Bmx, Btk, CHK (Csk homologous kinase), CSK (C-terminal Src Kinase), Itk-1, Src (c-Src, Lyn, Fyn, Lck, Syk, Hck, Yes, Blk, Fgr and Frk), Tec, Txk/Rlk, Abl, EGFR (EGFR-1/ErbB-1, ErbB-2/NEU/HER-2, ErbB-3 and ErbB-4), FAK, FGF1R (also FGFR1 or FGR-1), FGF2R (also FGR-2), MET (also Met-1 or c-MET), PDGFR-α, PDGFR-β, Tie-1, Tie-2 (also Tek-1 or Tek), VEGFRI (also FLT-1), VEGFR2 (also KDR), FLT-3, FLT-4, c-KIT, JAKI, JAK2, JAK3, TYK2, LOK, RET, TRKA, PYK2, ALK (Anaplastic Lymphoma Kinase), EPHA (1-8), EPHB (1-6), RON, Fes, Fer or EPHB4 (also EPHB4-1).

Examples of protein-serine/threonine kinases include, but are not limited to, Ark, ATM (1-3), CamK (I-IV), CamKK, Chk1 and 2 (Checkpoint kinases), CKI, CK2, Erk, IKK-I (also IKK-ALPHA or CHUK), IKK-2 (also IKK-BETA), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (A and B), PKA, P38, Erk (1-3), PKB (including all PKB subtypes) (also AKT-1, AKT-2, AKT-3 or AKT3-1), IRAK1, FRK, SGK, TAK1 or Tpl-2 (also COT).

Protein kinases play very important roles in the normal regulation of cell growth. However, as a result of either mutation or overexpression of the tyrosine kinases (receptor or non-receptor) or the ligands of the receptor tyrosine kinases, signaling can become deregulated, resulting in uncontrolled cell proliferation leading to cancer or a related disease, disorder or syndrome.

Protein kinases catalyze and regulate the process of phosphorylation, whereby the kinases covalently attach phosphate groups to proteins or lipid targets in response to a variety of extracellular signals: hormones, neurotransmitters, growth and differentiation factors, cell cycle events, environmental stresses, nutritional stresses and the like.

Phosphorylation modulates or regulates a variety of cellular processes such as proliferation, growth, differentiation, metabolism, apoptosis, motility, transcription, translation and other signaling processes. Uncontrolled signaling for cell growth due to defective control of protein phosphorylation has also been implicated in a number of diseases and disease conditions, such as osteoarthritis, rheumatoid arthritis, synovial pannus invasion in arthritis, multiple sclerosis, myasthenia gravis, diabetes mellitus, diabetic angiopathies or retinopathy, inflammatory bowel disease, Crohn's disease, ulcerative colitis, transplant or bone marrow transplant rejection, lupus, chronic pancreatitis, cachexia, septic shock, skin diseases or disorders (such as papilloma formation, psoriasis, dermatitis, eczema, seborrhea and the like), central nervous system diseases (such as Alzheimer's disease, Parkinson's disease, depression and the like), cancers (such as glioma cancers, epidermoid cancers, head and neck cancers, lung cancers, breast cancers, colorectal cancers, prostate cancers, gastric cancers, esophageal cancers or papillocarcinomas and the like and associated pathologies such as unregulated cell proliferation, tumor growth or vascularization or metastatic cancer cell invasion and migration and the like or leukemias or lymphomas), occular diseases (such as macular degeneration, diseases of the cornea, glaucoma and the like), viral infections (such as cytomegalovirus CMV), heart disease (such as atherosclerosis, neointima formation or transplantation-induced vasculopathies (such as restenosis and the like), lung or pulmonary diseases (such as allergic-asthma, lung fibrosis or complications resulting from chronic obstructive pulmonary disorder and the like) or kidney or renal diseases (such as acute, subacute or chronic forms of glomerulonephritis or membranoproliferative glomerulonephritis, glomerulosclerosis, congenital multicystic renal dysplasia, kidney fibrosis and the like). Therefore, kinase inhibitors have potential use as therapeutic agents.

The tyrosine kinases can further be categorized by whether they are receptor tyrosine kinases or non-receptor tyrosine kinases. The receptor tyrosine kinases span the cell membrane with a ligand interacting domain protruding from the cell, with a hydrophobic trans-membrane domain, and a cytoplasmic domain that contains the catalytic kinase domain and other regulatory sequences. Non-receptor tyrosine kinases are often myristylated or modified by the addition of other hydrophobic moieties that allow them to be anchored to the cell membrane.

Human cytomegalovirus (CMV) is a widespread opportunistic human herpes virus that causes severe and fatal diseases in those who are immune compromised and in transplant recipients. CMV is also a leading cause of atherosclerosis and virally mediated birth defects. The human CMV uses the EGFR receptor to enter cells during infection, EGFR is autophosphorylated and the downstream signal transduction pathway components are activated; however, the EGFR specific inhibitor tyrphostin AG1478 has been shown to reduce the viral load in cells that were infected in the presence of the tyrphostin (Wang, et al., Nature, 24 Jul. 2003, Vol 424). Accordingly, potent EGFR selective inhibitors may be useful in anti-CMV therapy.

Uncontrolled cell proliferation is the insignia of cancer. Cell proliferation in response to various stimuli is manifested by a deregulation of the cell division cycle, the process by which cells multiply and divide. Tumor cells typically have damage to the genes that directly or indirectly regulate progression through the cell division cycle.

Angiogenesis plays a role in various processes including development of the vasculature, wound healing and maintenance of the female reproductive system. Pathological angiogenesis is associated with disease states such as cancer, diabetic retinopathy, rheumatoid arthritis, endometriosis and psoriasis. Solid-tumor cancers, in particular, are dependent on angiogenesis for their growth. The vascular endothelial growth factors (VEGFs) are mediators of both normal and pathologic angiogenesis. VEGF transmits signals into cells through their cognate receptors, which belong to the receptor tyrosine kinase (RTK) family of transmembrane receptors. These receptors are tripartite, consisting of an extracellular ligand-binding domain, a transmembrane domain, which anchors the receptor in the membrane of the cell, and an intracellular tyrosine kinase domain.

One subfamily of RTKs comprises the receptors Flt1/VEGF-R1 and KDR/Flk1/VEGF-R2, which bind VEGFs. Binding of the VEGF ligand to the receptor results in stimulation of the receptor tyrosine kinase activity and transduction of biological signals into the cell. The KDR/Flk1/VEGF-R2 receptor mediates the biological activities of mitogenesis and proliferation of endothelial cells while the Flt1/VEGF-R1 receptor mediates functions such as endothelial cell adhesion. Inhibition of KDR/Flk1/VEGF-R2 signalling has been shown to inhibit the process of angiogenesis. Inhibitors of this receptor are likely useful in controlling or limiting angiogenesis.

Aurora kinases (Aurora-A, Aurora-B and Aurora-C) are highly conserved tyrosine kinases found in all organisms where they function to regulate microtubule dynamics during the M phase of the cell cycle and are essential for mitotic progression. Aurora-A kinase associates with the centrosome around the pericentriolar material, as well as the microtubules at the bipolar mitotic-spindle poles and the midbody microtubules and plays a role in spindle formation and organization of the centrosome. Aurora-B regulates chromosomal movement and cytokinesis and Aurora-C's biological function is not yet understood. The Aurora-A kinase is involved in centrosome separation, duplication and maturation as well as in bipolar spindle assembly and stability. Aurora-A is overexpressed in a number of different human cancers and tumor cell lines. Overexpression of Aurora is sufficient to induce growth in soft agar and transforms cells making them tumorigenic. Inhibition of Aurora activity results in centrosome/chromosome segregation defects leading to monopolar spindles and polyploidy which induces cell apoptosis in a variety of cancer cell lines and has suppressed tumor growth in vivo.

There is a need, for small-molecule compounds that may be readily synthesized and are potent inhibitors of one or more of VEGF-R2 kinase or Aurora-A kinase that possess anti-tumor cell proliferation activity, and as such are useful in treating or ameliorating a VEGF-R2 or Aurora-A kinase receptor mediated, angiogenesis-mediated or hyperproliferative disorder.

### SUMMARY OF THE INVENTION

Accordingly, one object of the invention is to attain compounds and drug compositions that inhibit the activity of one or more of the VEGF-R2 kinase or Aurora-A kinase receptors.

A further object is to provide an effective method of treating cancer indications and kinase mediated, angiogenesis-mediated or hyperproliferative disorders through VEGF-R2 kinase or Aurora-A kinase inhibition.

Another object is to achieve pharmaceutical compositions containing compounds effective to inhibit the proliferation of cancer cells.

These and other objects and advantages provided by the present invention will become apparent in light of the detailed description below and are achieved through use of a series of substituted dihydro-isoindolone compounds of formula (I): 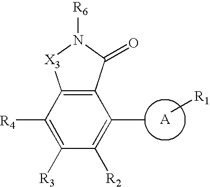
and forms thereof, wherein Ring A, X₃, R₁, R₂, R₃, R₄ and R₆ are as herein defined.

The present invention is further directed to compounds of formula (Ia): 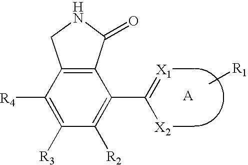
and forms thereof, wherein Ring A, X₁, X₂, R₁, R₂, R₃ and R₄ are as herein defined.

An example of the present invention includes using a compound of formula (I) as a protein kinase inhibitor.

An example of the present invention includes using a compound of formula (I) as an inhibitor of a tyrosine protein kinase such as VEGF-R2 or Aurora-A.

An example of the present invention includes a method for using a compound of formula (I) in treating or ameliorating a kinase mediated disorder associated with cellular proliferation or angiogenesis and the like.

An example of the present invention includes a method for using a compound of formula (I) as a therapeutic agent for treating, preventing or ameliorating a chronic or acute protein kinase mediated disease, disorder or condition in a subject in need thereof comprising administering to the subject an effective amount of a compound of formula (I) or composition thereof.

### DETAILED DESCRIPTION OF THE INVENTION

The present invention provides a compound of formula (I): 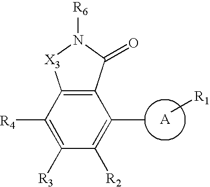
or a form thereof, whereinRing A is a heteroaromatic monocyclic or bicyclic ring system moiety;
X₃ is selected from the group consisting of CH₂ and C═O;
R₁ is one, two, three, four or five substituents each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
   wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
   wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
   wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₂, R₃ and R₄ is each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
   wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
   wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
   wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₅ is one, two, three, four or five substituents each selected from the group consisting of hydrogen, halogen, hydroxy, nitro, cyano, C₁₋₈alkyl, C₁₋₈alkoxy, C₁₋₈alkyl-halogen, C₁₋₈alkoxy-halogen, C₁₋₈alkyl-hydroxy, C₁₋₈alkoxy-hydroxy, amino, amino-C₁₋₈alkyl, C₁₋₁₈alkyl-amino and C₁₋₁₈alkyl-amino-C₁₋₈alkyl; and
R₆ is selected from the group consisting of hydrogen, optionally substituted C₁₋₈alkyl, C₃₋₈cycloalkyl-R₅, aryl-R₅, heteroaryl-R₅ and heterocyclyl-R₅,
   wherein C₁₋₈alkyl is optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅.

An example of the present invention is a compound of formula (I) or a pharmaceutically acceptable form thereof, wherein
Ring A is selected from the group consisting of pyrrol-2-yl, imidazol-2-yl, pyrazol-2-yl, indol-2-yl, indol-3-yl, isoindol-1-yl, and benzimidazol-2-yl;
R₁ is one or two substituents each selected from the group consisting of hydrogen, hydroxy, C₁₋₈alkoxy and carbonyl-C₁₋₈alkoxy, wherein C₁₋₈alkoxy is optionally substituted with hydroxy, aryl and heterocyclyl-R₅;
R₂, R₃ and R₄ is each selected from the group consisting of hydrogen, hydroxy, nitro, optionally substituted C₁₋₈alkoxy and optionally substituted amino, wherein amino is optionally substituted with one C₁₋₁₈acyl substituent;
R₅ is hydrogen or one substituent selected from C₁₋₈alkyl or C₁₋₈alkyl-hydroxy; and
R₆ is selected from the group consisting of hydrogen and C₁₋₈alkyl.

An example of the present invention is a compound of formula (I) or a pharmaceutically acceptable form thereof, wherein Ring A is selected from the group consisting of pyrrol-2-yl, imidazol-2-yl, pyrazol-2-yl, indol-2-yl, indol-3-yl, isoindol-1-yl, and benzimidazol-2-yl.

An example of the present invention is a compound of formula (I) or a pharmaceutically acceptable form thereof, wherein Ring A is selected from the group consisting of pyrrol-2-yl, indol-2-yl, indol-3-yl and benzimidazol-2-yl.

An example of the present invention is a compound of formula (I) or a pharmaceutically acceptable form thereof, wherein X₃ is CH₂.

An example of the present invention is a compound of formula (I) or a pharmaceutically acceptable form thereof, wherein X₃ is C═O.

An example of the present invention is a compound of formula (I) or a pharmaceutically acceptable form thereof, wherein R₁ is one or two substituents each selected from the group consisting of hydrogen, hydroxy, C₁₋₈alkoxy and carbonyl-C₁₋₈alkoxy, wherein C₁₋₈alkoxy is optionally substituted with hydroxy, aryl and heterocyclyl-R₅.

An example of the present invention is a compound of formula (I) or a pharmaceutically acceptable form thereof, wherein R₂, R₃ and R₄ is each selected from the group consisting of hydrogen, hydroxy, nitro, optionally substituted C₁₋₈alkoxy and optionally substituted amino, wherein amino is optionally substituted with one C₁₋₈acyl substituent.

An example of the present invention is a compound of formula (I) or a pharmaceutically acceptable form thereof, wherein R₅ is hydrogen.

An example of the present invention is a compound of formula (I) or a pharmaceutically acceptable form thereof, wherein R₅ is one substituent selected from C₁₋₈alkyl or C₁₋₈alkyl-hydroxy.

An example of the present invention is a compound of formula (I) or a pharmaceutically acceptable form thereof, wherein R6 is selected from the group consisting of hydrogen and C₁₋₈alkyl.

The present invention further provides a compound of formula (Ia): 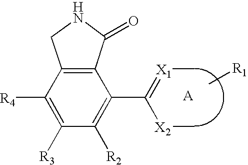
or a form thereof, whereinRing A is taken together with X₁ and X₂ to form a heteroaromatic monocyclic or bicyclic A(X₁,X₂) ring system moiety;
X₁ is selected from the group consisting of N and CH;
X₂ is selected from the group consisting of NH, CH and CH₂; wherein X₁ and X₂ cannot simultaneously be CH and CH₂, respectively;
R₁ is one, two, three, four or five substituents each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
   wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
   wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
   wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₂, R₃ and R₄ is each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
   wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
   wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
   wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₅ is one, two, three, four or five substituents each selected from the group consisting of hydrogen, halogen, hydroxy, nitro, cyano, C₁₋₈alkyl, C₁₋₈alkoxy, C₁₋₈alkyl-halogen, C₁₋₈alkoxy-halogen, C₁₋₈alkyl-hydroxy, C₁₋₈alkoxy-hydroxy, amino, amino-C₁₋₈alkyl, C₁₋₈alkyl-amino and C₁₋₈alkyl-amino-C₁₋₈alkyl.

An example of the present invention is a compound of formula (Ia) or a pharmaceutically acceptable form thereof, wherein the A(X₁,X₂) ring system moiety is selected from the group consisting of pyrrol-2-yl, imidazol-2-yl, pyrazol-2-yl, indol-2-yl, isoindol-1-yl and benzimidazol-2-yl.

An example of the present invention is a compound of formula (Ia) or a pharmaceutically acceptable form thereof, wherein the A(X₁,X₂) ring system moiety is selected from the group consisting of pyrrol-2-yl, indol-2-yl and benzimidazol-2-yl.

An example of the present invention is a compound of formula (Ia) or a pharmaceutically acceptable form thereof, wherein R₁ is one, two, three, four or five substituents each selected from the group consisting of hydrogen, halogen, hydroxy, optionally substituted C₁₋₈alkyl, optionally substituted C₁₋₈alkoxy, optionally substituted amino, optionally substituted carbamoyl and carbonyl-C₁₋₈alkoxy,
wherein C₁₋₈alkyl and C₁₋₈alkoxy is each optionally substituted with one substituent selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino-C₁₋₈alkyl, C₁₋₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy.

An example of the present invention is a compound of formula (Ia) or a pharmaceutically acceptable form thereof, wherein R₁ is one or two substituents selected from the group consisting of hydrogen, halogen, hydroxy, optionally substituted C₁₋₈alkoxy, optionally substituted amino, optionally substituted carbamoyl and carbonyl-C₁₋₈alkoxy,
wherein C₁₋₈alkoxy is optionally substituted with one substituent selected from the group consisting of hydroxy, amino, amino-C₁₋₈alkyl, amino-C₁₋₈alkyl-C₁₋₈alkoxy, aryl-R₅, and heterocyclyl-R₅,
wherein amino is optionally substituted with one or two substituents each selected from the group consisting of C₁₋₈alkyl, C₁₋₈alkyl-amino-C₁₋₈alkyl, C₁₋₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl-amino-C₁₋₈alkyl and C₁₋₈alkyl-C₁₋₈alkoxy.

An example of the present invention is a compound of formula (Ia) or a pharmaceutically acceptable form thereof, wherein R₁ is one or two substituents selected from the group consisting of hydrogen, hydroxy, optionally substituted C₁₋₈alkoxy; and carbonyl-C₁₋₈alkoxy, wherein C₁₋₈alkoxy is optionally substituted with one substituent selected from the group consisting of hydroxy, aryl, and heterocyclyl-R₅.

An example of the present invention is a compound of formula (Ia) or a pharmaceutically acceptable form thereof, wherein R₂, R₃ and R₄ is each selected from the group consisting of hydrogen, halogen, hydroxy, nitro, optionally substituted C₁₋₁₈alkyl, optionally substituted C₁₋₈alkoxy, optionally substituted amino, optionally substituted carbamoyl and carbonyl-C₁₋₈alkoxy,
wherein C₁₋₈alkyl and C₁₋₈alkoxy is each optionally substituted with one, two, three, four or five substituents each selected from the group consisting of hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino-C₁₋₈alkyl, C₁₋₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino-C₁₋₈alkyl and C₁₋₈alkyl-C₁₋₈alkoxy.

An example of the present invention is a compound of formula (Ia) or a pharmaceutically acceptable form thereof, wherein R₂, R₃ and R₄ is each selected from the group consisting of hydrogen, halogen, hydroxy, nitro, C₁₋₈alkyl, optionally substituted C₁₋₈alkoxy, optionally substituted amino, optionally substituted carbamoyl and carbonyl-C₁₋₈alkoxy,
wherein C₁₋₈alkoxy is optionally substituted with one, two, three, four or five substituents each selected from the group consisting of hydroxy, amino, amino-C₁₋₈alkyl and amino-C₁₋₈alkyl-C₁₋₈alkoxy,
wherein amino is optionally substituted with one or two substituents each selected from the group consisting of C₁₋₈alkyl-amino-C₁₋₈alkyl, C₁₋₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl and C₁₋₈alkyl-amino-C₁₋₈alkyl.

An example of the present invention is a compound of formula (Ia) or a pharmaceutically acceptable form thereof, wherein R₂, R₃ and R₄ is each selected from the group consisting of hydrogen, halogen, hydroxy, nitro, C₁₋₈alkyl, optionally substituted C₁₋₈alkoxy, optionally substituted amino, and carbonyl-C₁₋₈alkoxy,
wherein C₁₋₈alkoxy is optionally substituted with one substituent selected from the group consisting of hydroxy, amino, amino-C₁₋₈alkyl and amino-C₁₋₈alkyl-C₁₋₈alkoxy; and
wherein amino is optionally substituted with one substituent selected from the group consisting of C₁₋₁₈alkyl, C₁₋₁₈alkyl-amino-C₁₋₈alkyl, C₁₋₈acyl, and C₁₋₈acyl-amino-C₁₋₈alkyl.

An example of the present invention is a compound of formula (Ia) or a pharmaceutically acceptable form thereof, wherein R₂, R₃ and R₄ is each selected from the group consisting of hydrogen, hydroxy, nitro, C₁₋₈alkoxy, and optionally substituted amino, wherein amino is optionally substituted with C₁₋₈acyl.

An example of the present invention is a compound of formula (Ia) or a pharmaceutically acceptable form thereof, wherein R₅ is one substituent selected from the group consisting of hydrogen, halogen, C₁₋₁₈alkyl, C₁₋₈alkyl-halogen, C₁₋₈alkyl-hydroxy, C₁₋₈alkyl-amino and C₁₋₈alkyl-amino-C₁₋₈alkyl.

An example of the present invention is a compound of formula (Ia) or a pharmaceutically acceptable form thereof, wherein R₅ is one substituent selected from the group consisting of hydrogen, halogen, C₁₋₈alkyl and C₁₋₈alkyl-hydroxy.

An example of the present invention is a compound of formula (Ib): 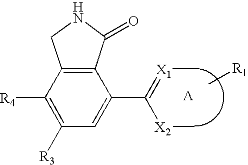
or a form thereof, wherein
the A(X₁,X₂) ring system moiety is selected from the group consisting of pyrrol-2-yl, indol-2-yl and benzimidazol-2-yl;
R₁ is one substituent selected from the group consisting of hydrogen, hydroxy, optionally substituted C₁₋₈alkoxy and carbonyl-C₁₋₈alkoxy, wherein C₁₋₈alkoxy is optionally substituted with one substituent selected from the group consisting of hydroxy, aryl-R₅, and heterocyclyl-R₅;
R₃ is selected from the group consisting of hydrogen, hydroxy, and C₁₋₈alkoxy;
R4 is selected from the group consisting of hydrogen, nitro and optionally substituted amino, wherein amino is optionally substituted with C₁₋₈acyl; and
R₅ is one substituent selected from the group consisting of hydrogen, C₁₋₈alkyl and C₁₋₈alkyl-hydroxy.

An example of the present invention is a compound of formula (I), wherein A(X₁,X₂)—R₁, X₃, R₃, R₄, and R₆ are each dependently selected from:

| Cpd | A(X₁,X₂)—R₁ | X₃ | R₃ | R₄ | R₆ |
|---|---|---|---|---|---|
| 1 | 5-[O(CH₂)₃-4-CH₂CH₃-piperazin-1- | CH₂ | H | H | H |
| | yl]-indol-2-yl | | | | |
| 2 | 5-[O(CH₂)₃-4-CH₂OH-piperidin-1- | CH₂ | H | H | H |
| | yl]-indol-2-yl | | | | |
| 3 | 5-OCH₃-indol-2-yl | CH₂ | H | H | H |
| 4 | 5-OH-indol-2-yl | CH₂ | H | H | H |
| 5 | 5-O(CH₂)₃OH-indol-2-yl | CH₂ | H | H | H |
| 6 | 5-OCH₃-indol-2-yl | CH₂ | OCH₃ | H | H |
| 7 | indol-2-yl | CH₂ | H | H | H |
| 8 | 5-[O(CH₂)₂-morpholin-4-yl]-indol-2-yl | CH₂ | H | H | H |
| 9 | 5-[O(CH₂)₃-piperidin-1-yl]-indol-2-yl | CH₂ | H | H | H |
| 10 | 5-[O(CH₂)₂-piperidin-1-yl]-indol-2-yl | CH₂ | H | H | H |
| 11 | 5-OCH₃-benzimidazol-2-yl | CH₂ | H | H | H |
| 12 | pyrrol-2-yl | CH₂ | H | H | H |
| 13 | 1-C(O)OC(CH₃)₃-5-(benzyloxy)- | CH₂ | OH | H | H |
| | indol-2-yl | | | | |
| 14 | 5-(benzyloxy)-indol-2-yl | CH₂ | OH | H | H |
| 15 | 1-C(O)OC(CH₃)₃-5-(OCH₃)-indol-2-yl | CH₂ | H | NO₂ | H |
| 16 | 5-OCH₃-indol-2-yl | CH₂ | H | NO₂ | H |
| 17 | 5-OCH₃-indol-2-yl | CH₂ | H | NH₂ | H |
| 18 | 5-OCH₃-indol-2-yl | CH₂ | H | NHC(O)CH₃ | H |
| 19 | 5-(benzyloxy)-indol-2-yl | CH₂ | H | H | H |
| 20 | indol-3-yl | CH₂ | H | H | H |
| 21 | 1-C(O)OC(CH₃)₃-5-(OCH₃)-indol-2-yl | C═O | H | H | H |
| 22 | 5-OCH₃-indol-2-yl | C═O | H | H | H |
| 23 | 1-C(O)OC(CH₃)₃-pyrrol-2-yl | CH₂ | H | H | H |
| 24 | 1-C(O)OC(CH₃)₃-pyrrol-2-yl | CH₂ | H | H | CH₃ |
| 25 | pyrrol-2-yl | CH₂ | H | H | CH₃ |
| 26 | 1-C(O)OC(CH₃)₃-indol-2-yl | CH₂ | H | H | H |
| 27 | 1-C(O)OC(CH₃)₃-5-(OCH₃)-indol-2-yl | CH₂ | H | H | H |
| 28 | 1-C(O)OC(CH₃)₃-5-OH-indol-2-yl | CH₂ | H | H | H |
| 29 | 1-C(O)OC(CH₃)₃-5-[O(CH₂)₂- | CH₂ | H | H | H |
| | piperidin-1-yl]-indol-2-yl | | | | |
| 30 | 1-C(O)OC(CH₃)₃-5-[O(CH₂)₂- | CH₂ | H | H | H |
| | morpholin-4-yl]-indol-2-yl | | | | |
| 31 | 1-C(O)OC(CH₃)₃-5-OCH₃-indol-2-yl | CH₂ | OCH₃ | H | H |
| 32 | 1-C(O)OC(CH₃)₃-5-(benzyloxy)- | CH₂ | H | H | H |
| | indol-2-yl | | | | |
| 33 | 1-C(O)OC(CH₃)₃-5-(benzyloxy)- | CH₂ | H | NO₂ | H |
| | indol-2-yl | | | | |

An example of the present invention is a compound of formula (I) or a form thereof represented by a compound selected from:

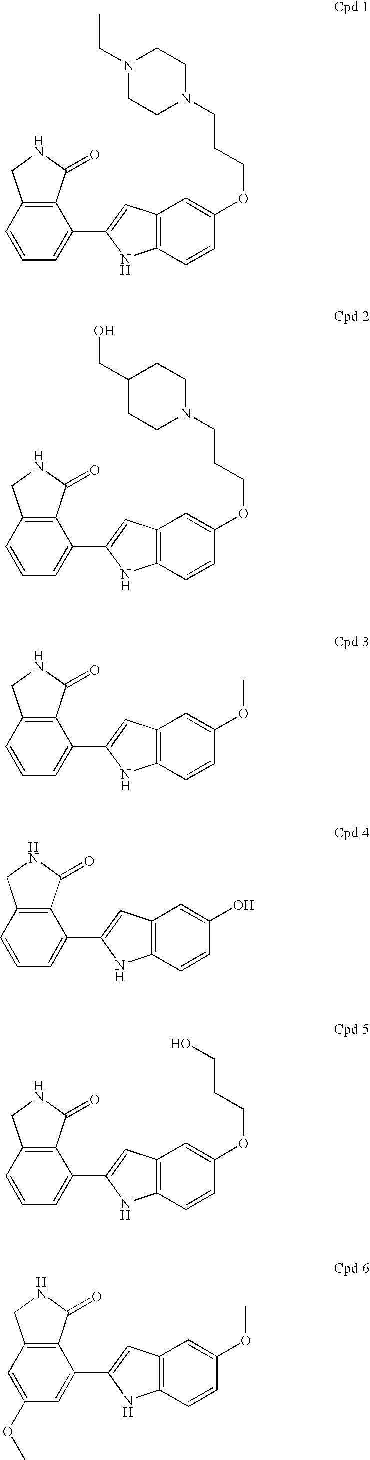

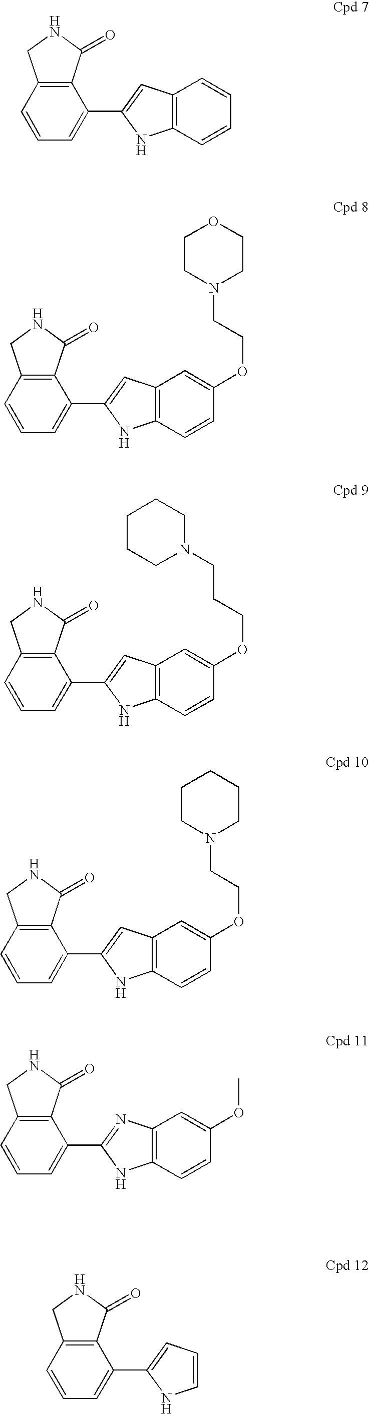

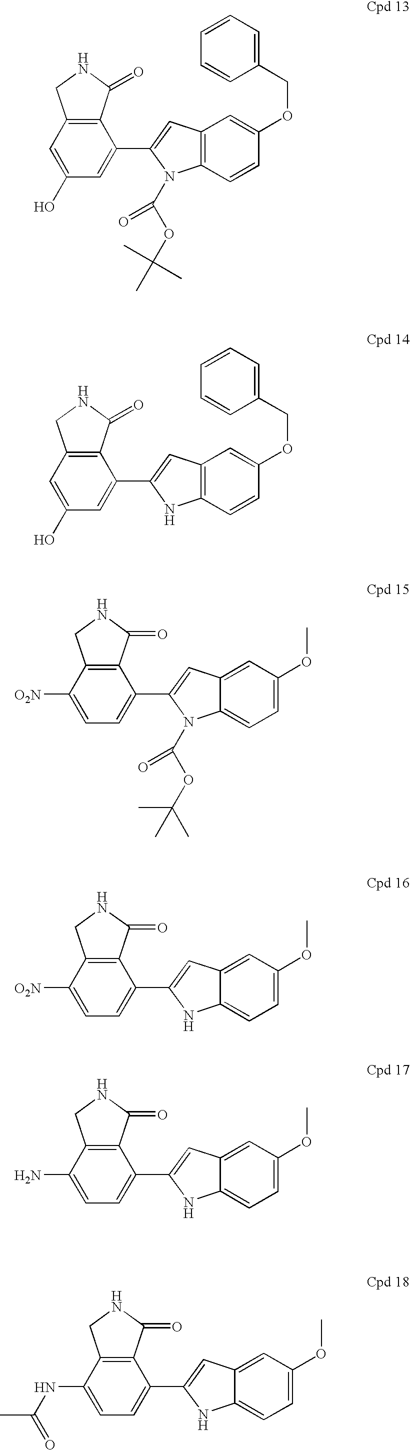

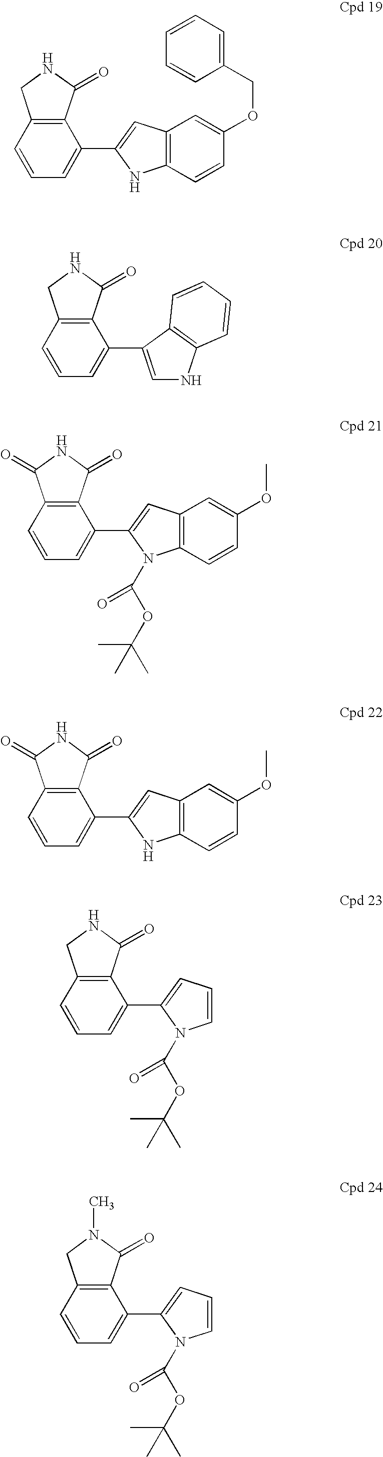

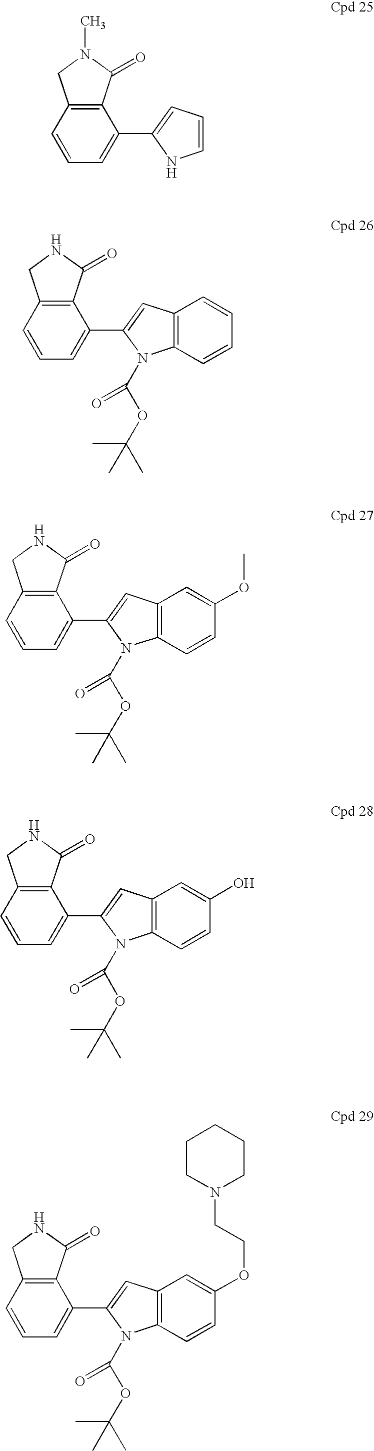

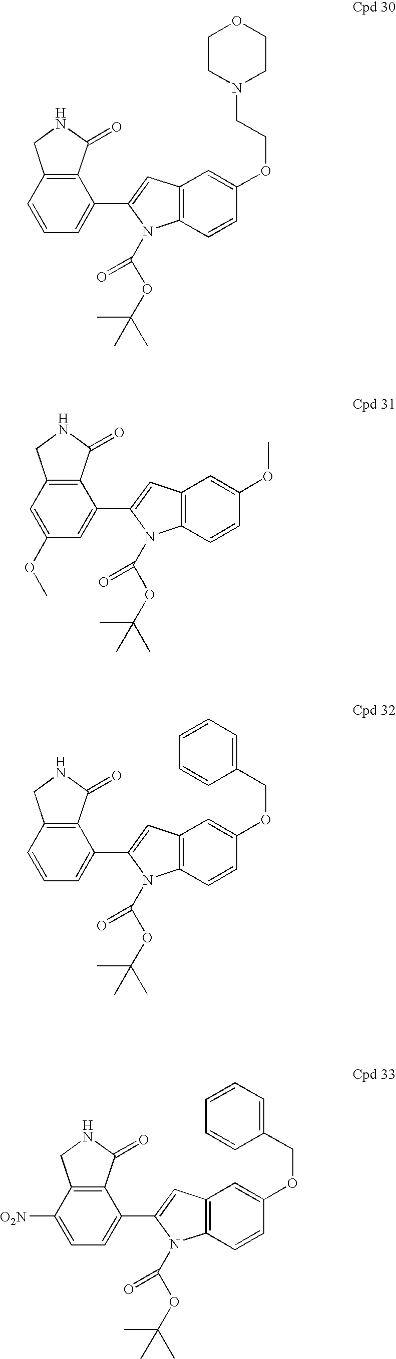

An example of the present invention is a compound of formula (I), wherein the compound is a VEGF-R2 kinase or Aurora-A kinase inhibitor.

The present invention provides a method for using compounds of formula (I) in treating or ameliorating a kinase receptor-mediated disorder.

An example of the method includes inhibiting unregulated kinase activity comprising contacting the kinase domain with one or more compounds of formula (I).

An example of the method includes inhibiting a kinase by contacting the kinase receptor with a compound of formula (I).

An example of the method includes inhibiting increased or unregulated kinase expression or signaling leading to unregulated cell proliferation comprising contacting a kinase receptor with one or more compounds of formula (I).

An embodiment of the invention is a compound of formula (I), wherein the compound is a VEGF-R2 kinase or Aurora-A kinase inhibitor.

The present invention also provides a method for using the compounds of formula (I) in treating or ameliorating VEGF-R2 kinase or Aurora-A kinase mediated disorder.

An embodiment of the invention is a method for using a compound of formula (I) for treating or ameliorating a VEGF-R2 kinase or Aurora-A kinase mediated disorder.

An example of the method includes inhibiting a cyclin dependent kinase by contacting the kinase receptor with a compound of formula (I).

An example of the method includes inhibiting the unregulated expression of a cyclin dependent kinase and the like.

An embodiment of the invention is a method for inhibiting a kinase selected from the group consisting of VEGF-R2 kinase or Aurora-A kinase by contacting the kinase receptor with a compound of formula (I).

An embodiment of the invention is a method for treating or ameliorating a kinase mediated disorder in a subject in need thereof comprising administering to the subject an effective amount of a compound of formula (I).

Another embodiment of the invention is a method for treating or ameliorating a VEGF-R2 kinase or Aurora-A kinase mediated disorder in a subject in need thereof comprising administering to the subject an effective amount of a compound of formula (I) for treating or ameliorating a VEGF-R2 kinase or Aurora-A kinase mediated disorder.

Chemical Definitions

As used herein, the following terms have the following meanings:

The term “C₁₋₈alkyl,” whether used alone or as part of a substituent group, means a saturated branched or straight chain monovalent hydrocarbon radical or alkyldiyl linking group having a specified number of carbon atoms, wherein the radical is derived by the removal of one hydrogen atom from a single carbon atom and the alkyldiyl linking group is derived by the removal of one hydrogen atom from each of two carbon atoms in the chain. The term “C₁₋₈alkyl” refers to a radical having from 1-8 carbon atoms in a linear or branched arrangement. Typical alkyl radicals include, but are not limited to, methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Embodiments include, e.g., the alkyl groups C₁₋₈alkyl or C₁₋₄alkyl. Alkyl and alkyldiyl radicals may be attached to a core molecule via a terminal carbon atom or via a carbon atom within the chain. Similarly, any number of substituent variables may be attached to an alkyl or alkyldiyl radical when allowed by available valences.

The term “C₁₋₈alkoxy,” whether used alone or as part of a substituent group, means an alkyl or alkyldiyl alcohol radical derived by the removal of the hydrogen atom from the hydroxide oxygen portion of the alcohol radical. Typical embodiments include, e.g., the alkoxy groups C₁₋₈alkoxy or C₁₋₄alkoxy. For example, “C₁₋₈alkoxy” specifically includes the radicals methoxy, ethoxy, propoxy, butoxy, pentoxy, hexoxy, heptoxy, octoxy and the like. As described above, an alkoxy radical may be similarly attached to a core molecule and further substituted where indicated.

The term “C₃₋₈cycloalkyl,” whether used alone or as part of a substituent group, means a saturated or partially unsaturated cyclic hydrocarbon ring system. Typical cycloalkyl radicals include, but are not limited to, cyclopropyl, cyclobutyl, cyclopentyl, cyclohexyl, indanyl, fluorenyl, acenaphthenyl and the like.

The term “heterocyclyl,” whether used alone or as part of a substituent group, means a saturated or partially unsaturated cyclic ring radical derived by the removal of one hydrogen atom from a single carbon atom of the ring system and in which one or more ring carbon atoms are a heteroatom selected from N, O, S, SO or SO₂. Embodiments include monocyclic or bicyclic rings wherein 1, 2, 3 or 4 members of the ring are a nitrogen atom, or 0, 1, 2 or 3 members of the ring are nitrogen atoms and 1 member is an oxygen or sulfur atom.

Typical heterocyclyl radicals include, and are not limited to, dihydro-1H-pyrrole (including 2-pyrrolinyl or 3-pyrrolinyl), pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, pyran, tetrahydropyranyl, tetrahydrothiopyranyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furyl, tetrahydro-thienyl, tetrahydro-pyranyl, tetrahydro-pyridazinyl, 1,3-benzodioxol-5-yl, 2,3-dihydro-1,4-benzodioxin-6-yl and the like.

The term “aryl,” whether used alone or as part of a substituent group, means an unsaturated cyclic ring radical derived by the removal of one hydrogen atom from a single carbon atom of the ring system. Typical aryl radicals include, and are not limited to, phenyl, naphthalenyl, indenyl, azulenyl, anthracenyl, biphenyl and the like.

The term “heteroaryl,” whether used alone or as part of a substituent group, means an unsaturated cyclic ring radical derived by the removal of one hydrogen atom from a single carbon atom of the ring system and in which one or more ring carbon atoms are a heteroatom selected from N, O, S, SO or SO₂.

Typical heteroaryl radicals include, and are not limited to, furyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzoxazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term oyl, when used as a suffix to a ring system (e.g. aroyl, heteroaroyl or heterocycloyl), means a radical of the formula: —C(O)-ring system.

The term C₁₋₈acyl means a radical of the formula: —C(O)—C₁₋₈alkyl; or, when used as a prefix to a ring system (e.g. C₁₋₈acyl-C₃₋₈cycloalkyl, C₁₋₁₈acyl-aryl, C₁₋₈acyl-heteroaryl or C₁₋₈acyl-heterocyclyl), means a radical of the formula: —C(O)—C₁₋₈alkyl-ring system.

The term C₁₋₈acyl-amino-C₁₋₈alkyl means a radical of the formula: —C(O)—C₁₋₈alkyl-NH—C₁₋₈alkyl or —C(O)—C₁₋₈alkyl-N(C₁₋₈alkyl)₂.

The term C₁₋₈alkyl-C₁₋₈alkoxy means a radical of the formula: —C₁₋₈alkyl-O—C₁₋₈alkyl.

The term C₁₋₈alkyl-amino means a radical of the formula: —C₁₋₈alkyl-NH₂.

The term C₁₋₈alkyl-amino-C₁₋₈alkyl means a radical of the formula: —C₁₋₈alkyl-NH—C₁₋₈alkyl or —C₁₋₈alkyl-N(C₁₋₈alkyl)₂.

The term C₁₋₈alkyl-halogen means a radical of the formula: —C₁₋₈alkyl(halo)₁₋₃ and includes monofluoromethyl, difluoromethyl, trifluoromethyl, trifluoroethyl and the like.

The term C₁₋₈alkyl-hydroxy means a radical wherein C₁₋₈alkyl is substituted on an available carbon chain atom with one or more hydroxy radicals.

The term C₁₋₈alkoxy-halogen means a radical of the formula: —O—C₁₋₈alkyl(halo)₁₋₃ and includes monofluoromethoxy, difluoromethoxy, trifluoromethoxy, trifluoroethoxy and the like.

The term C₁₋₈alkoxy-hydroxy means a radical wherein —O—C₁₋₈alkyl is substituted on an available carbon chain atom with one or more hydroxy radicals.

The term amino means a radical of the formula: —NH₂.

The term amino-C₁₋₈-alkyl means a radical of the formula: —NH—C₁₋₈alkyl or N(C₁₋₈alkyl)₂.

The term amino-C₁₋₈alkyl-C₁₋₈alkoxy means a radical of the formula: —NH—C₁₋₈alkyl-O—C₁₋₈alkyl or —N[(C₁₋₈alkyl)(C₁₋₁₈alkyl-O—C₁₋₈alkyl)].

The term carbamoyl means a radical of the formula: —C(O)NH₂.

The term carbonyl-C₁₋₈-alkoxy means a radical of the formula: —C(O)—O—C₁₋₈alkyl.

The term halogen means the group chloro, bromo, fluoro or iodo.

The term “substituted” means the independent replacement of one or more hydrogen atoms within a radical with that amount of substitutents allowed by available valences.

The term “dependently substituted” means that the structure variables are specified in an indicated combination.

In general, IUPAC nomenclature rules are used throughout this disclosure.

Compound Forms

The term “forms” and “forms thereof” means that the compounds of the present invention may exist in various salt, stereoisomer, crystalline, solvate, ester, prodrug or active metabolite forms. The present invention encompasses all such compound forms, including active compounds in the form of essentially pure enantiomers, racemic mixtures and tautomers.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Pharmaceutically acceptable acidic/anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide salts.

Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol, ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate, sodium hydroxide, triethanolamine or zinc.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” means isomers of identical constitution that differ in the spatial arrangement of their atoms. Enantiomers and diastereomers are stereoisomers wherein an asymmetrically substituted carbon atom acts as a chiral center. The term “chiral” means a molecule that is not superimposable on its mirror image, implying the absence of an axis and a plane or center of symmetry. The term “enantiomer” means one of a pair of molecular species that are mirror images of each other and are not superimposable. The term “diastereomer” means stereoisomers that are not related as mirror images. The symbols “R” and “S” represent the configuration of substituents around a chiral carbon atom(s).

The term “racemate” or “racemic mixture” means a compound of equimolar quantities of two enantiomeric species, wherein the compound is devoid of optical activity. The term “optical activity” means the degree to which a chiral molecule or non-racemic mixture of chiral molecules rotates the plane of polarized light.

“Geometric isomer” means isomers that differ in the orientation of substituent atoms in relationship to a carbon-carbon double bond, to a cycloalkyl ring, or to a bridged bicyclic system. Substituent atoms (other than H) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

The isomeric descriptors (“R.” “S,” “E,” and “Z”) indicate atom configurations relative to a core molecule and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the invention may have one or more polymorph or amorphous crystalline forms. Said forms are included in the scope of the invention. In addition, some of the compounds may form solvates with water (i.e., hydrates) or common organic solvents. Said solvates are encompassed within the scope of this invention.

Methods of Use

The present invention includes a first method for inhibiting unregulated protein kinase activity comprising contacting a protein kinase domain with one or more compounds of formula (I).

The first method also includes inhibiting unregulated serine-threonine and tyrosine protein kinase activity.

The first method also includes inhibiting increased or unregulated protein kinase expression or signaling leading to unregulated cell proliferation.

The first method further comprises inhibiting the unregulated expression of a protein kinase such as VEGF-R2, Aurora-A and the like.

The present invention includes a second method for use of one or more compounds of formula (I) as a therapeutic agent for treating, preventing or ameliorating a chronic or acute protein kinase mediated disease, disorder or condition in a subject in need thereof comprising administering to the subject an effective amount of one or more compounds of formula (I) or a pharmaceutical composition thereof.

The second method includes use as a therapeutic agent for inhibiting the effects of unregulated kinase activity in the subject.

The second method includes use as a therapeutic agent for treating, preventing or ameliorating a chronic or acute kinase mediated disease, disorder or condition associated with cellular proliferation or angiogenesis and the like in the subject.

The second method includes use as a therapeutic agent for treating, preventing or ameliorating a chronic or acute kinase mediated cancer in the subject.

The second method includes use as a therapeutic agent for suppressing a chronic or acute tumor associated with non-small-cell lung cancers, colon cancers, breast cancers and the like.

The second method also includes treating, preventing or ameliorating chronic unregulated cell proliferation whereby cancer remission is induced in the subject.

The second method includes treating, preventing or ameliorating a chronic or acute serine-threonine or tyrosine protein kinase mediated disease, disorder or condition in the subject.

The second method includes treating, preventing or ameliorating a chronic or acute VEGF-R2, Aurora-A and the like protein kinase mediated disease, disorder or condition in the subject.

The second method includes treating or preventing a chronic or acute kinase mediated disease, disorder or condition characterized by unregulated cell proliferation or metastatic cancer cell invasion and migration in the subject.

The second method includes administering to the subject an effective amount of a compound of formula (I) or composition thereof in the form of a medicament. Consequently, the invention encompasses the use of the compound of formula (I) as a medicament.

The present invention includes a third method for use of a compound of formula (I) as a marker, wherein the compound is labeled with a ligand such as a radioligand (selected from deuterium, tritium and the like).

The present invention includes a fourth method for treating or ameliorating chemotherapy induced alopecia in a subject in need thereof comprising topically administering to the subject an effective amount of a compound of formula (I) or pharmaceutical composition thereof.

The present invention includes the use of a compound of formula (I) for the manufacture of a medicament for treating any of the diseases, disorders or conditions mentioned in any of the foregoing methods.

The term “chronic or acute kinase mediated disease, disorder or condition” as used herein, includes, and is not limited to diseases, disorders or conditions associated with unregulated kinase activity and conditions that accompany such activity.

The term “unregulated kinase activity” refers to 1) increased or unregulated kinase expression or signaling, 2) increased kinase expression leading to unregulated cell proliferation, 3) increased kinase signalling leading to unregulated cell proliferation, or 4) mutations leading to constitutive kinase activation. The existence of unregulated kinase activity may be determined by procedures well known in the art.

The term “unregulated cell proliferation” refers to cell proliferation of one or more subset of cells in a multicellular organism resulting in harm (such as discomfort or decreased life expectancy) to the multicellular organism.

Tumor cells which result from unregulated cell proliferation use many mechanisms to enhance their survival and spread and often have high rates of proliferation because growth control signals that keep normal cells in check are defective. Many tumor cells secrete autocrine growth factors that increase proliferation rates or they induce other cells to secrete growth factors that they utilize.

Tumor cells grow and spread by dislodging from a primary tumor site, using proteases to digest the extracellular matrix, spreading in response to migration cues, allowing them to migrate to certain tissues preferentially where overexpressed adhesion molecules allow attachment and growth at the new site. The totality of these and other biological processes are responsible for the lethal effects of a tumor. A kinase inhibitor may affect one or more aspects of tumor survival mechanisms and thus be therapeutically useful. Alternatively, a kinase inhibitor may not affect one particular tumor survival mechanism but may still be therapeutically useful by affecting tumor survival by an unknown or as yet unelucidated mechanism of action.

The term “treating, preventing or ameliorating” includes, and is not limited to, facilitating the eradication of, inhibiting the progression of or promoting stasis of a malignancy.

The foregoing methods contemplate that the compounds of the present invention are therapeutically useful for treating, preventing or ameliorating kinase mediated diseases, disorders or conditions such as, without limitation, the kinase mediated disorder is selected from osteoarthritis, rheumatoid arthritis, synovial pannus invasion in arthritis, multiple sclerosis, myasthenia gravis, diabetes mellitus, diabetic angiopathies or retinopathy, inflammatory bowel disease, Crohn's disease, ulcerative colitis, transplant or bone marrow transplant rejection, lupus, chronic pancreatitis, cachexia, septic shock, skin diseases or disorders (selected from papilloma formation, psoriasis, dermatitis, eczema, seborrhea, chemotherapy-induced alopecia), central nervous system diseases (selected from neuronal apoptosis, Alzheimer's disease, Parkinson's disease or depression), mycotic infection, an acute or chronic cancer (selected from glioma cancers, epidermoid cancers, head and neck cancers, lung cancers, breast cancers, colorectal cancers, prostate cancers, gastric cancers, esophageal cancers, papillocarcinomas, associated pathologies such as unregulated cell proliferation, tumor growth, tumor vascularization, angiopathy, angiogenesis, metastatic cancer cell invasion and migration, leukemias or lymphomas), occular diseases (selected from macular degeneration, diseases of the cornea, glaucoma or neovascular glaucoma), viral infections (selected from cytomegalovirus), heart disease (selected from atherosclerosis, neointima formation or transplantation-induced vasculopathies (selected from restenosis), lung or pulmonary diseases (selected from allergic-asthma, lung fibrosis or complications resulting from chronic obstructive pulmonary disorder) or kidney or renal diseases (selected from acute, subacute or chronic forms of glomerulonephritis or membranoproliferative glomerulonephritis, glomerulosclerosis, congenital multicystic renal dysplasia or kidney fibrosis).

An embodiment of the method of the present invention includes kinase mediated disorders selected from mycotic infection, cancer, tumor growth, tumor vascularization, angiopathy, angiogenesis, chemotherapy-induced alopecia or restenosis.

The term “administering,” with respect to the methods of the present invention, refers to a means for treating, ameliorating or preventing a disease, disorder or condition as described herein with a compound specifically disclosed or a compound or prodrug thereof, which would obviously be included within the scope of the invention albeit not specifically disclosed for certain of the instant compounds.

Such methods include prophylactically or therapeutically administering an effective amount of one or more compounds of formula (I) or a composition or medicament thereof at different times during the course of a therapy or concurrently in a combination form. Prophylactic administration can occur prior to the manifestation of symptoms characteristic of a kinase associated disease or disorder such that the disease or disorder is prevented or, alternatively, delayed in its progression. The instant invention is therefore to be understood as embracing all such regimes of simultaneous or alternating treatment and the term “administering” is to be interpreted accordingly.

The term “prodrug” refers to a metabolic precursor of a compound of formula (I) or pharmaceutically acceptable form thereof. In general, a prodrug is a functional derivative of a compound which may be inactive when administered to a subject but is readily convertible in vivo into an active metabolite compound.

The term “active metabolite” refers to a metabolic product of a compound that is pharmaceutically acceptable and effective. Conventional procedures for the selection and preparation of suitable prodrug derivatives are described, for example, in “Design of Prodrugs”, ed. H. Bundgaard, Elsevier, 1985.

The term “subject” as used herein, refers to a patient, such as an animal, preferably a mammal, most preferably a human, who has been the object of treatment, observation or experiment and is at risk of (or susceptible to) developing a disease or disorder or having a disease or disorder related to unregulated kinase activity.

The term “effective amount” refers to that amount of active compound or pharmaceutical agent that elicits the biological or medicinal response (such as inhibiting activation of unregulated kinase activity) in a tissue system, animal or human, that is being sought by a researcher, veterinarian, medical doctor, or other clinician, which includes treating, preventing or ameliorating the symptoms of the disease, disorder or condition being treated.

The effective amount of a compound of formula (I) exemplified in such a method is from about 0.001 mg/kg/day to about 300 mg/kg/day or has an IC₅₀ (50% inhibition concentration) against protein kinase activity in a range of about 25 μM or less, of about 10 μM or less, of about 1 μM or less, of about 0.5 μM or less, of about 0.25 μM or less or of about 0.1 μM or less.

The term “composition” refers to a product containing a compound of the present invention (such as a product comprising the specified ingredients in the specified amounts, as well as any product which results, directly or indirectly, from such combinations of the specified ingredients in the specified amounts).

The term “medicament” refers to a product for use in treating, preventing or ameliorating a kinase mediated disease, disorder or condition.

The term “pharmaceutically acceptable” refers to molecular entities and compositions that are of sufficient purity and quality for use in the formulation of a composition or medicament of the present invention and that, when appropriately administered to an animal or a human, do not produce an adverse, allergic or other untoward reaction. Since both human use (clinical and over-the-counter) and veterinary use are equally included within the scope of the present invention, a pharmaceutically acceptable formulation would include a composition or medicament for either human or veterinary use.

The methods of the present invention further include administering to the subject an effective amount of a combination product comprising one or more compounds of formula (I) or a composition or medicament thereof and at least one other therapeutic agent at different times during the course of a therapy or concurrently as a combination product.

Such a combination product may advantageously facilitate administering to the subject an amount of an agent or a compound of formula (I) that is either or both reduced relative to the amount which would be given in the absence of the other.

Therefore, it is contemplated that the compounds of this invention can be administered to the subject before, during or after the time a particular therapeutic agent is administered The term “therapeutic agent” includes, and is not limited to, chemotherapeutic agents to treat cancer such as anti-angiogenic agents, anti-tumor agents, cytotoxic agents, inhibitors of cell proliferation and the like.

The term “combination therapy” refers to the use of one or more compounds of formula (I) or composition or medicament thereof advantageously administered in one or more cell anti-proliferation therapies including chemotherapy, radiation therapy, gene therapy or immunotherapy or as an adjunct to chemotherapy and radiation therapy for treating, preventing or ameliorating a chronic or acute protein kinase mediated disease, disorder or condition.

The combination therapy comprises
1. coadministration of a compound of formula (I) or pharmaceutical composition thereof and a chemotherapeutic agent for treating, preventing or ameliorating a chronic or acute protein kinase mediated disease, disorder or condition,
2. sequential administration of a compound of formula (I) or pharmaceutical composition thereof and a chemotherapeutic agent for treating, preventing or ameliorating a chronic or acute protein kinase mediated disease, disorder or condition,
3. administration of a pharmaceutical composition containing a compound of formula (I) or pharmaceutical composition thereof and a chemotherapeutic agent for treating, preventing or ameliorating a chronic or acute protein kinase mediated disease, disorder or condition, or,
4. simultaneous administration of a separate pharmaceutical composition containing a compound of formula (I) or pharmaceutical composition thereof and a separate pharmaceutical composition containing a chemotherapeutic agent for treating, preventing or ameliorating a chronic or acute protein kinase mediated disease, disorder or condition.

For example, an inhibitor compound of the present invention, acting as an anti-angiogenic agent can be administered in a dosing regimen with at least one other cytotoxic compound, such as a DNA alkylating agent.

Preferred anti-tumor agents are selected from the group consisting of cladribine (2-chloro-2′-deoxy-(beta)-D-adenosine), chlorambucil (4-(bis(2-chlorethyl)amino)benzenebutanoic acid), DTIC-Dome (5-(3,3-dimethyl-1-triazeno)-imidazole-4-carboxamide), platinum chemotherapeutics and nonplatinum chemotherapeutics.

Platinum containing anti-tumor agents include, and are not limited to, cisplatin (CDDP) (cis-dichlorodiamineplatinum).

Non-platinum containing anti-tumor agents include, and are not limited to, adriamycin (doxorubicin), aminopterin, bleomycin, camptothecin, carminomycin, combretastatin(s), cyclophosphamide, cytosine arabinoside, dactinomycin, daunomycin, epirubicin, etoposide (VP-16), 5-fluorouracil (5FU), herceptin actinomycin-D, methotrexate, mitomycin C, tamoxifen, taxol, taxotere, thiotepa, vinblastine, vincristine, vinorelbine and derivatives and prodrugs thereof.

Each anti-tumor agent is administered in an effective amount, which varies based on the agent used, the type of malignancy to be treated or ameliorated and other conditions according to methods well known in the art.

As will be understood by those of ordinary skill in the art, the appropriate doses of chemotherapeutic agents will be generally around those already employed in clinical therapies wherein the chemotherapeutics are administered alone or in combination with other chemotherapeutics.

By way of example only, agents such as cisplatin, and other DNA alkylating are used widely to treat cancer. The efficacious dose of cisplatin used in clinical applications is about 20 mg/m² for 5 days every three weeks for a total of three courses. Cisplatin is not absorbed orally and must therefore be delivered via injection intravenously, subcutaneously, intratumorally or intraperitoneally.

Further useful agents include compounds that interfere with DNA replication, mitosis and chromosomal segregation. Such chemotherapeutic agents include adriamycin (doxorubicin), etoposide, verapamil or podophyllotoxin and the like and are widely used in clinical settings for tumor treatment. These compounds are administered through bolus injections intravenously at doses ranging from about 25 to about 75 mg/m² at 21 day intervals (for adriamycin) or from about 35 to about 50 mg/m² (for etoposide) intravenously or at double the intravenous dose orally.

Agents that disrupt the synthesis and fidelity of polynucleotide precursors such as 5-fluorouracil (5-FU) are preferentially used to target tumors. Although quite toxic, 5-FU is commonly used via intravenous administration with doses ranging from about 3 to about 15 mg/kg/day.

The method of the present invention further includes a method for administering a compound of the present invention in combination with radiation therapy. As used herein, “radiation therapy” refers to a therapy comprises exposing the subject in need thereof to radiation. Such therapy is known to those skilled in the art. The appropriate scheme of radiation therapy will be similar to those already employed in clinical therapies wherein the radiation therapy is used alone or in combination with other chemotherapeutics.

The method of the present invention further includes a method for administering a compound of the present invention in combination with a gene therapy. As used herein, “gene therapy” refers to a therapy targeting on particular genes involved in tumor development. Possible gene therapy strategies include the restoration of defective cancer-inhibitory genes, cell transduction or transfection with antisense DNA corresponding to genes coding for growth factors and their receptors, or with the so-called ‘suicide genes’.

The method of the present invention further includes a method for administering a compound of the present invention in combination with an immunotherapy. As used herein, “immunotherapy” refers to a therapy targeted to a particular protein involved in tumor development via antibodies specific to such protein. For example, monoclonal antibodies against vascular endothelial growth factor have been used in treating cancers.

An example of the present invention includes a method for treating, preventing or ameliorating a chronic or acute protein kinase mediated disease, disorder or condition, particularly a tumor, in a subject in need thereof comprising administering to the subject an effective amount of a compound of formula (I) or pharmaceutical composition thereof conjugated to a targeting agent and delivered or “seeded” directly or indirectly into tissues with unregulated kinase activity.

The term “delivered or “seeded” directly or indirectly into tissues” includes conjugating a compound of formula (I) to a targeting agent which then directs the conjugate to its intended site of action (i.e., to vascular endothelial cells or to tumor cells). The term “targeting agent” includes the use of both antibody and non-antibody agents. Because of the specific interaction between the targeting agent and its corresponding binding partner, a compound of this invention can be administered with high local concentrations at or near a target site and thus treats the disorder at the target site more effectively.

An antibody targeting agent includes antibodies or antigen-binding fragments thereof, that bind to a targetable or accessible component of a tumor cell, tumor vasculature or tumor stroma. The “targetable or accessible component” of a tumor cell, tumor vasculature or tumor stroma, is preferably a surface-expressed, surface-accessible or surface-localized component. The antibody targeting agents also include antibodies or antigen-binding fragments thereof, that bind to an intracellular component that is released from a necrotic tumor cell. Preferably such antibodies are monoclonal antibodies or antigen-binding fragments thereof that bind to insoluble intracellular antigen(s) present in cells that may be induced to be permeable or in cell ghosts of substantially all tumor or normal cells, but are not present or accessible on the exterior of normal living cells of a mammal.

As used herein, the term “antibody” is intended to refer broadly to any immunologic binding agent such as IgG, IgM, IgA, IgE, F(ab′)2, a univalent fragment such as Fab′, Fab, Dab, as well as engineered antibodies such as recombinant antibodies, humanized antibodies, bispecific antibodies, and the like. The antibody can be either the polyclonal or the monoclonal, although the monoclonal is preferred. There is a very broad array of antibodies known in the art that have immunological specificity for the cell surface of virtually any solid tumor type (see a Summary Table on monoclonal antibodies for solid tumors in U.S. Pat. No. 5,855,866 (Thorpe, et al). Methods are known to those skilled in the art to produce and isolate antibodies to be used as targeting agents against tumors (U.S. Pat. No. 5,855,866 (Thorpe); and, U.S. Pat. No. 6,342,219 (Thorpe)).

Non-antibody targeting agents include growth factors, such as PDGF, VEGF and FGF; peptides containing the tripeptide R-G-D, that bind specifically to the tumor vasculature and other targeting components such as annexins and related ligands. In addition, a variety of other organic molecules can also be used as targeting agents for tumors, examples are hyaluronan oligosaccharides which specifically recognize Hyaluronan-binding protein, a cell surface protein expressed during tumor cell and endothelial cell migration and during capillary-like tubule formation (U.S. Pat. No. 5,902,795 (Toole, et al.)) and polyanionic compounds, particularly polysulphated or polysulphonated compounds such as N- and O-sulfated polyanionic polysaccharides, polystyrene sulfonate and other polyanionic compounds (as described in U.S. Pat. No. 5,762,918 (Thorpe) which selectively bind to vascular endothelial cells.

Techniques for conjugating therapeutic moiety to antibodies are well known (Amon, et al., Monoclonal Antibodies For Immunotargeting Of Drugs In Cancer Therapy, Monoclonal Antibodies And Cancer Therapy, Reisfeld, et al. (eds.), pp. 243-56 (Alan R. Liss, Inc. 1985); Hellstrom, et al., Antibodies For Drug Delivery, Controlled Drug Delivery (2nd Ed.), Robinson, et al. (eds.), pp. 623-53 (Marcel Dekker, Inc. 1987); Thorpe, Antibody Carriers Of Cytotoxic Agents In Cancer Therapy: A Review, Monoclonal Antibodies '84: Biological And Clinical Applications, Pinchera, et al. (eds.), pp. 475-506 (1985). Similar techniques can also be applied to attach compounds of the invention to non-antibody targeting agents. Those skilled in the art will know or be able to determine methods of forming conjugates with non-antibody targeting agents, such as oligopeptides, polysaccharides or other polyanionic compounds.

Although any linking moiety that is reasonably stable in blood can be used to link the compound of the invention to the targeting agent, those with biologically-releasable bonds and/or selectively cleavable spacers or linkers are preferred. “Biologically-releasable bonds” and “selectively cleavable spacers or linkers” refers to those linking moieties which have reasonable stability in the circulation and are releasable, cleavable or hydrolyzable only or preferentially under certain conditions, (i.e., within a certain environment or in contact with a particular agent). Such bonds include, for example, disulfide and trisulfide bonds and acid-labile bonds (as described in U.S. Pat. Nos. 5,474,765 and 5,762,918) and enzyme-sensitive bonds, including peptide bonds, esters, amides, phosphodiesters and glycosides (as described in U.S. Pat. Nos. 5,474,765 and 5,762,918). Such selective-release design features facilitate sustained release of the compounds from the conjugates at the intended target site.

The effective amount of a compound of the invention conjugated to a targeting agent depends on the individual, the disease type, the disease state, the method of administration and other clinical variables. The effective amount is readily determinable using data from an animal model. Experimental animals bearing solid tumors are frequently used to optimize appropriate therapeutically effective amount prior to translating to a clinical environment. Such models are known to be very reliable in predicting effective anti-cancer strategies. For example, mice bearing solid tumors are widely used in pre-clinical testing to determine working ranges of therapeutic agents that give beneficial anti-tumor effects with minimal toxicity.

The present invention further provides a pharmaceutical composition that comprises an effective amount of the compound of the invention conjugated to a targeting agent and a pharmaceutically acceptable carrier. When proteins such as antibodies or growth factors, or polysaccharides are used as targeting agents, they are preferably administered in the form of injectable compositions. The injectable antibody solution will be administered into a vein, artery or into the spinal fluid over the course of from about 2 minutes to about 45 minutes, preferably from about 10 to about 20 minutes. In certain cases, intradermal and intracavitary administration are advantageous for tumors restricted to areas close to particular regions of the skin and/or to particular body cavities. In addition, intrathecal administrations may be used for tumors located in the brain.

Another aspect of the present invention includes a method for treating or disorders related to unregulated kinase activity (in particular, restenosis, intimal hyperplasia or inflammation in vessel walls) in a subject in need thereof comprising administering to the subject by controlled delivery an effective amount of a compound of formula (I) or pharmaceutical composition thereof coated onto a intraluminal medical device (in particular, a balloon-catheter or stent). Such devices are useful to prevent the occurrence of restenosis by inhibiting upregulated kinase activity and thus preventing hyperproliferation of the endothelium.

The term “intraluminal medical device” refers to any delivery device, such as intravascular drug delivery catheters, wires, pharmacological stents and endoluminal paving. It is preferred that the delivery device comprises a stent that includes a coating or sheath which elutes or releases the compounds. The term “controlled delivery” refers to the release of active ingredient in a site-directed and time dependent manner. Alternatively, the delivery system for such a device may comprise a local infusion catheter that delivers the compound at a variably controlled rate.

The term “stent” refers to any device capable of being delivered by a catheter. A stent is routinely used to prevent vascular closure due to physical anomalies such as unwanted inward growth of vascular tissue due to surgical trauma. A stent often has a tubular, expanding lattice-type structure appropriate to be left inside the lumen of a duct to relieve an obstruction. The stent has a lumen wall-contacting surface and a lumen-exposed surface. The lumen-wall contacting surface is the outside surface of the tube and the lumen-exposed surface is the inner surface of the tube. The stent material may be a polymeric, metallic or a combination polymeric-metallic material and can be optionally biodegradable.

The compounds of the present invention can also be administered in the form of liposome delivery systems, such as small unilamellar vesicles, large unilamellar vesicles and multilamellar vesicles. Liposomes containing delivery systems as well known in the art are formed from a variety of phospholipids, such as cholesterol, stearylamine or phosphatidylcholines.

Pharmaceutical Compositions

An example of the present invention includes a pharmaceutical composition comprising an admixture of one or more compounds of formula (I) and/or one or more pharmaceutically acceptable forms thereof and one or more pharmaceutically acceptable excipients.

The pharmaceutically acceptable forms for a compound of formula (I) include a pharmaceutically acceptable salt, ester, prodrug or active metabolite of a compound of formula (I).

Pharmaceutical compositions according to the invention may, alternatively or in addition to a compound of formula I, comprise as an active ingredient a pharmaceutically acceptable salt of a compound of formula I or a prodrug or pharmaceutically active metabolite of such a compound or salt.

The present invention further includes the use of a process for making the composition or medicament comprising mixing one or more of the instant compounds and an optional pharmaceutically acceptable carrier; and, includes those compositions or medicaments resulting from such a process. Contemplated processes include both conventional and unconventional pharmaceutical techniques.

The composition or medicament may take a wide variety of forms to effectuate mode of administration, including, but not limited to, intravenous (both bolus and infusion), oral, nasal, transdermal, topical with or without occlusion, and injection intraperitoneally, subcutaneously, intramuscularly, intratumorally or parenterally. The composition or medicament may be in a dosage unit such as a tablet, pill, capsule, powder, granule, sterile parenteral solution or suspension, metered aerosol or liquid spray, drop, ampoule, auto-injector device or suppository; for administration orally, parenterally, intranasally, sublingually or rectally or by inhalation or insufflation.

Compositions or medicaments suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for parenteral administration include sterile solutions, emulsions and suspensions. Furthermore, compositions or medicaments can be administered in intranasal form via topical use of suitable intranasal vehicles, or via transdermal routes, using, e.g., those forms of transdermal skin patches well known to those of ordinary skill in that art.

Advantageously, a compound of formula (I) may be administered in a single daily dose, or the total daily dosage may be administered in divided doses of two, three or four times daily. Alternatively, the composition or medicament may be presented in a form suitable for once-weekly or once-monthly administration; for example, an insoluble salt of the active compound, such as the decanoate salt, may be adapted to provide a depot preparation for intramuscular injection.

The dosage form (tablet, capsule, powder, injection, suppository, teaspoonful and the like) containing the composition or medicament contains an effective amount of the active ingredient necessary to be therapeutically or prophylactically effective as described above.

The composition or medicament may contain from about 0.001 mg to about 5000 mg (preferably, from about 0.001 to about 500 mg) of the active compound or prodrug thereof and may be constituted into any form suitable for the mode of administration selected for a subject in need. A contemplated effective amount may range from about 0.001 mg to about 300 mg/kg of body weight per day. Preferably, the range is from about 0.003 to about 100 mg/kg of body weight per day. Most preferably, the range is from about 0.005 to about 15 mg/kg of body weight per day. The composition or medicament may be administered according to a dosage regimen of from about 1 to about 5 times per day.

For oral administration, the composition or medicament is preferably in the form of a tablet or capsule containing, e.g., 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated.

Optimal dosages will vary depending on factors associated with the particular subject being treated (e.g., age, weight, diet and time of administration), the severity of the condition being treated, the compound being employed, the mode of administration and the strength of the preparation. The use of either daily administration or post-periodic dosing may be employed.

A representative compound of formula (I) or a form thereof for use in the therapeutic methods and pharmaceutical compositions, medicines or medicaments described herein includes a compound selected from the group consisting of:

| Cpd | Name |
|---|---|
| 1 | 7-{5-[3-(4-ethyl-piperazin-1-yl)-propoxy]-1H-indol-2-yl}-2,3-dihydro-isoindol-1- |
| | one, |
| 2 | 7-{5-[3-(4-hydroxymethyl-piperidin-1-yl)-propoxy]-1H-indol-2-yl}-2,3-dihydro- |
| | isoindol-1-one, |
| 3 | 7-(5-methoxy-1H-indol-2-yl)-2,3-dihydro-isoindol-1-one, |
| 4 | 7-(5-hydroxy-1H-indol-2-yl)-2,3-dihydro-isoindol-1-one, |
| 5 | 7-[5-(3-hydroxy-propoxy)-1H-indol-2-yl]-2,3-dihydro-isoindol-1-one, |
| 6 | 5-methoxy-7-(5-methoxy-1H-indol-2-yl)-2,3-dihydro-isoindol-1-one, |
| 7 | 7-(1H-indol-2-yl)-2,3-dihydro-isoindol-1-one, |
| 8 | 7-[5-(2-morpholin-4-yl-ethoxy)-1H-indol-2-yl]-2,3-dihydro-isoindol-1-one, |
| 9 | 7-[5-(3-piperidin-1-yl-propoxy)-1H-indol-2-yl]-2,3-dihydro-isoindol-1-one, |
| 10 | 7-[5-(2-piperidin-1-yl-ethoxy)-1H-indol-2-yl]-2,3-dihydro-isoindol-1-one, |
| 11 | 7-(5-methoxy-1H-benzoimidazol-2-yl)-2,3-dihydro-isoindol-1-one, |
| 12 | 7-(1H-pyrrol-2-yl)-2,3-dihydro-isoindol-1-one, |
| 13 | 5-benzyloxy-2-(6-hydroxy-3-oxo-2,3-dihydro-1H-isoindol-4-yl)-indole-1-carboxylic |
| | acid tert-butyl ester, |
| 14 | 7-(5-benzyloxy-1H-benzoimidazol-2-yl)-6-hydroxy-2,3-dihydro-isoindol-1-one, |
| 15 | 5-methoxy-2-(7-nitro-3-oxo-2,3-dihydro-1H-isoindol-4-yl)-indole-1-carboxylic acid |
| | tert-butyl ester, |
| 16 | 7-(5-methoxy-1H-indol-2-yl)-4-nitro-2,3-dihydro-isoindol-1-one, |
| 17 | 4-amino-7-(5-methoxy-1H-indol-2-yl)-2,3-dihydro-isoindol-1-one, |
| 18 | N-[7-(5-methoxy-1H-indol-2-yl)-1-oxo-2,3-dihydro-1H-isoindol-4-yl]-acetamide, |
| 19 | 7-(5-benzyloxy-1H-indol-2-yl)-2,3-dihydro-isoindol-1-one, |
| 20 | 7-(1H-Indol-3-yl)-2,3-dihydro-isoindol-1-one, |
| 21 | 2-(1,3-dioxo-2,3-dihydro-1H-isoindol-4-yl)-5-methoxy-indole-1-carboxylic acid |
| | tert-butyl ester, |
| 22 | 4-(5-methoxy-1H-indol-2-yl)-isoindole-1,3-dione, |
| 23 | 2-(3-oxo-2,3-dihydro-1H-isoindol-4-yl)-pyrrole-1-carboxylic acid tert-butyl ester, |
| 24 | 2-(2-methyl-3-oxo-2,3-dihydro-1H-isoindol-4-yl)-pyrrole-1-carboxylic acid tert- |
| | butyl ester, |
| 25 | 2-methyl-7-(1H-pyrrol-2-yl)-2,3-dihydro-isoindol-1-one, |
| 26 | 2-(3-oxo-2,3-dihydro-1H-isoindol-4-yl)-indole-1-carboxylic acid tert-butyl ester, |
| 27 | 5-methoxy-2-(3-oxo-2,3-dihydro-1H-isoindol-4-yl)-indole-1-carboxylic acid tert- |
| | butyl ester, |
| 28 | 5-hydroxy-2-(3-oxo-2,3-dihydro-1H-isoindol-4-yl)-indole-1-carboxylic acid tert- |
| | butyl ester, |
| 29 | 2-(3-oxo-2,3-dihydro-1H-isoindol-4-yl)-5-(3-piperidin-1-yl-ethoxy)-indole-1- |
| | carboxylic acid tert-butyl ester, |
| 30 | 5-(2-morpholin-4-yl-ethoxy)-2-(3-oxo-2,3-dihydro-1H-isoindol-4-yl)-indole-1- |
| | carboxylic acid tert-butyl ester, |
| 31 | 5-Methoxy-2-(6-methoxy-3-oxo-2,3-dihydro-1H-isoindol-4-yl)-indole-1-carboxylic |
| | acid tert-butyl ester, |
| 32 | 5-benzyloxy-2-(3-oxo-2,3-dihydro-1H-isoindol-4-yl)-indole-1-carboxylic acid tert- |
| | butyl ester, and |
| 33 | 5-benzyloxy-2-(7-nitro-3-oxo-2,3-dihydro-1H-isoindol-4-yl)-indole-1-carboxylic |
| | acid tert-butyl ester. |

Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. Except where indicated, starting materials and intermediates used in the schemes and examples are prepared by known methodologies well within the ordinary skill of persons versed in the art. No attempt has been made to optimize the yields obtained in any of the example reactions. One skilled in the art would also know how to increase such yields through routine variations in materials, solvents, reagents, reaction conditions and the like. All commercially available chemicals were used without further purification. Particular equipment components used in the examples such as reaction vessels and the like are also commercially available.

The terms used in describing the invention are commonly used and known to those skilled in the art. When used herein, the following abbreviations have the indicated meanings:

| Abbreviation | Meaning |
|---|---|
| Boc | tert-butoxycarbonyl; tert-butyl ester |
| AIBN | 2,2′-azobisisobutyronitrile |
| Cpd | compound |
| DDBPP | dicyclohexyl-(2′,6′-dimethoxy-biphenyl-2-yl)-phosphane |
| DCM | dichloromethane |
| DMF | N,N-dimethylformamide |
| DMSO | dimethyl sulfoxide |
| EDCI | N-(3-dimethylaminopropyl)-N′-ethyl carbodiimide |
| EtOAc | ethyl acetate |
| EtOH | ethanol |
| hr(s)/min(s) | hour(s)/min(s) |
| LiOH | lithium hydroxide |
| MeOH | methanol |
| NBS | N-bromo-succinimide |
| NH₄OH | ammonium hydroxide |
| RT/rt/r.t. | room temperature |
| sat'd | saturated |
| TBAF | tert-butyl ammonium fluoride |
| TEA or Et₃N | triethylamine |
| TFA | trifluoroacetic acid |
| THF | tetrahydrofuran |
| TMSCHN₂ | trimethylsilyl diazomethane; also known as diazomethyl- |
| | trimethyl-silane |

General Synthetic Methods

Representative compounds of the present invention can be synthesized in accordance with the general synthetic methods described below, which are illustrated more particularly in the schemes that follow. The invention should not be construed as being limited by the chemical reactions and conditions expressed.

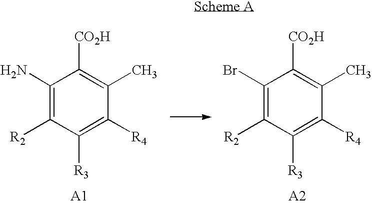

A solution of a Compound A1 is brominated at a suitable temperature with a suitable reagent solution (such as copper(I) bromide, hydrobromic acid, sodium nitrite and the like or mixtures thereof in water) to provide a Compound A2.

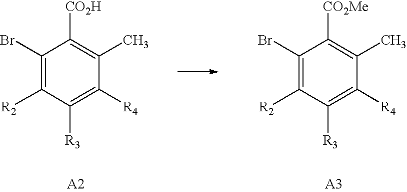

Compound A2 is reacted with an alkylating agent (such as TMSCHN₂ and the like) to provide a Compound A3.

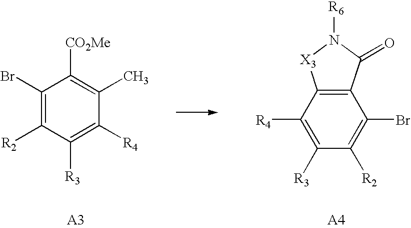

Compound A3 is reacted with a solution of a suitable reagent or a mixture thereof (such as AIBN and N-bromo-succinimide and the like) in the presence of an amine reagent (such as ammonium hydroxide or a substituted amine and the like) to provide a Compound A4 (as described in Rupert K C, Dodd J H and Henry J R, Heterocycles, 1997, 2217-2221).

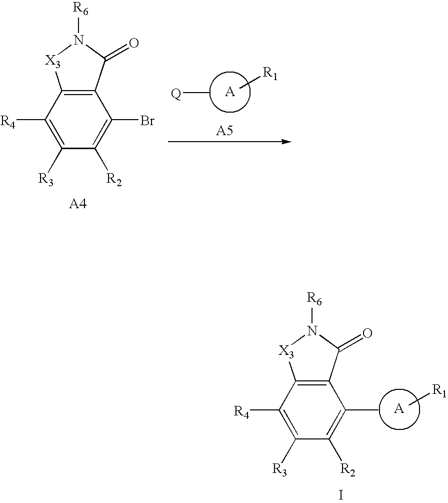

Compound A4 is reacted with a Compound A5 (wherein Q is a boronic acid or ester and the like) in the presence of a palladium catalyst (such as Pd(OAc)₂, Pd(dppf)₂Cl₂, Pd(PPh₃)₄ and the like) to provide a compound of formula (I).

Alternatively, Compound A4 is reacted with a boronating reagent (such as boronic acid or ester such as bis(pinacolato)diboron and the like) to form a boronated intermediate amenable for further reaction with a bromine substituted Compound A5 (wherein Q is bromine and the like) to thus provide a compound of formula (I).

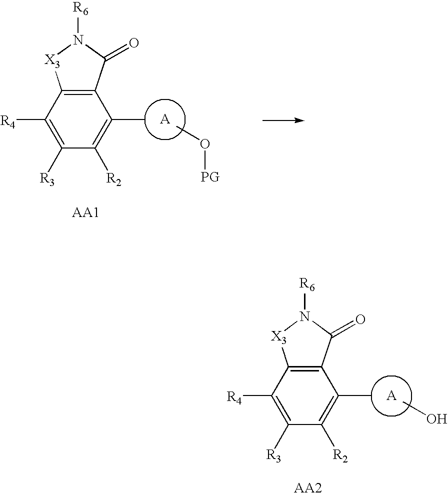

A solution of Compound AA1 (in a solvent such as THF) (wherein the A Ring is substituted with —O-PG, representing a protected alkoxy group, wherein PG is benzyloxy or tert-butyldimethylsilyloxy) is reacted with an appropriate deprotecting reagent (such as H₂ in the presence of Pd on charcoal in a solvent such as EtOH or TBAF) to provide a Compound AA2.

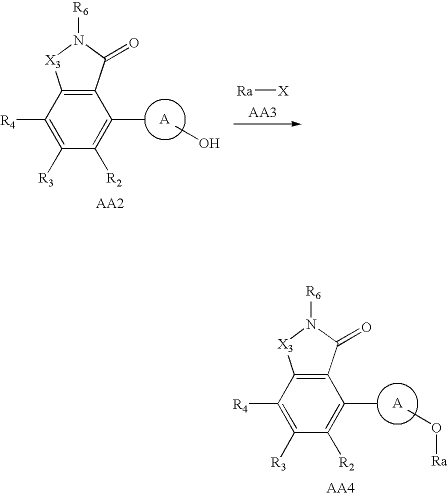

Compound AA2 is reacted with a Compound AA3 (wherein X is Cl, Br, I, SO₃C₆H₄CH₃, SO₃CH₃ or SO₃CF₃ and when R₁ is optionally substituted C₁₋₈alkoxy, Ra is the C₁₋₈alkyl portion of C₁₋₈alkoxy) and a suitable base (such as NaH, t-BuOK or Et₃N) in a solvent (such as THF or DMF) to provide a Compound AA4.

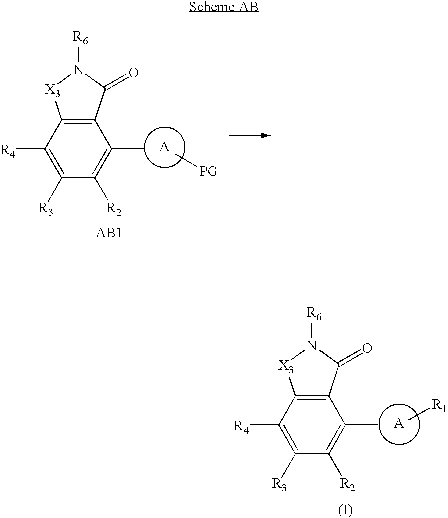

Compound AB1 (wherein the A Ring is substituted with PG, representing a protecting group such as benzyloxy, tert-butyloxycarbonyl or benzenulfonyl) is reacted with an appropriate deprotecting reagent (such as H₂ in the presence Pd on charcoal, trifluoroacetic acid or aqueous hydroxide) in a solvent (such as EtOH or THF) to provide a compound of formula (I). Alternatively, Compound AB1 is heated without solvent under N₂ to provide a compound of formula (I).

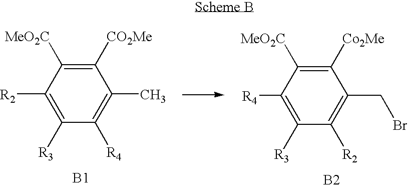

A solution of Compound B1 (in a solvent such as CCl₄) is brominated at a suitable temperature with a suitable reagent (such as NBS) in the presence of AIBN to provide a Compound B2.

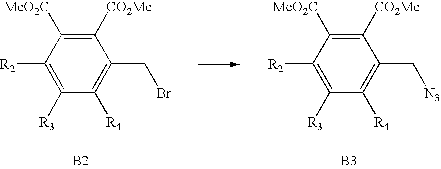

Compound B2 is reacted with an azide salt such as NaN₃ in a suitable solvent such as DMF to provide a Compound B3.

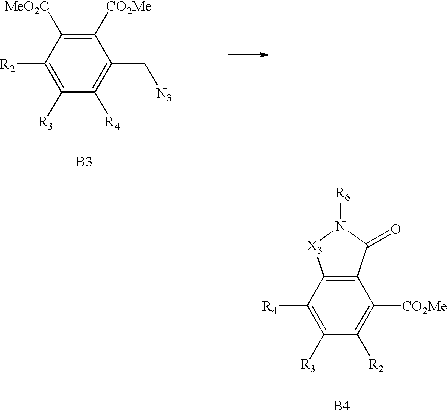

Compound B3 is treated with a reducing agent such as Ph₃P in a suitable solvent such as aqueous THF to provide a cyclized Compound B4.

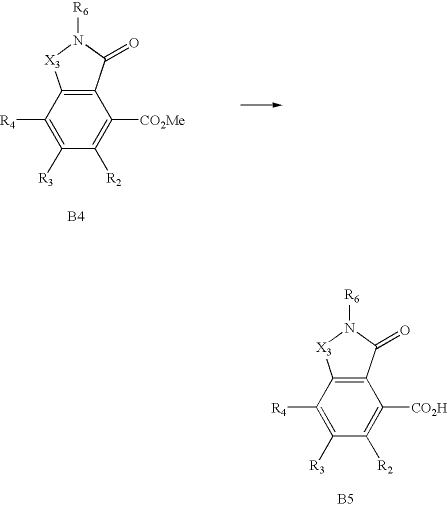

Compound B4 is hydrolyzed by treatment with a suitable base such as LiOH in a suitable solvent mixture such as MeOH, THF and water under refluxing conditions to afford a Compound B5.

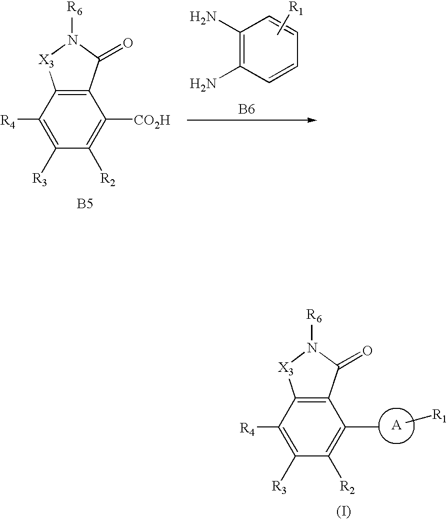

Compound B5 is combined with a suitable diamine Compound B6 and the mixture is cyclized under acidic conditions (by using an acid such as 6N HCl) to provide a compound of formula (I), wherein Ring A is benzimidazol-2-yl and R₆ is hydrogen.

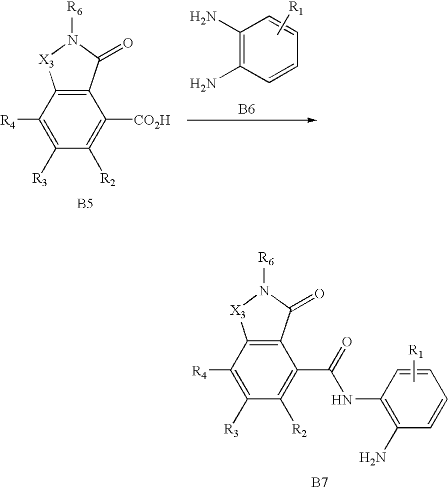

Alternatively, Compound B5 is coupled with a suitable diamine Compound B6 using a coupling reagent (such as EDCI in a suitable solvent such as DMF) to provide a Compound B7.

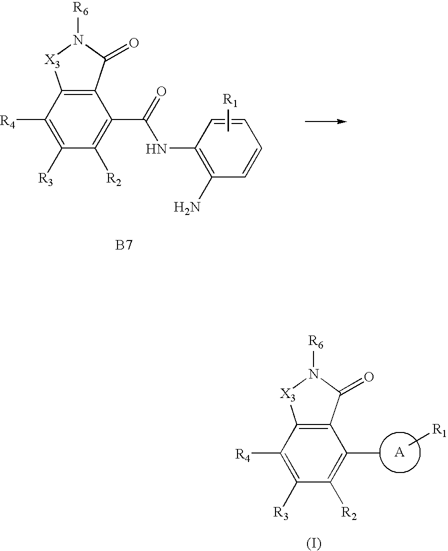

Compound B7 is cyclized by heating in a suitable solvent (such as acetic acid) to provide a compound of formula (I) wherein Ring A is benzimidazol-2-yl and R₆ is hydrogen.

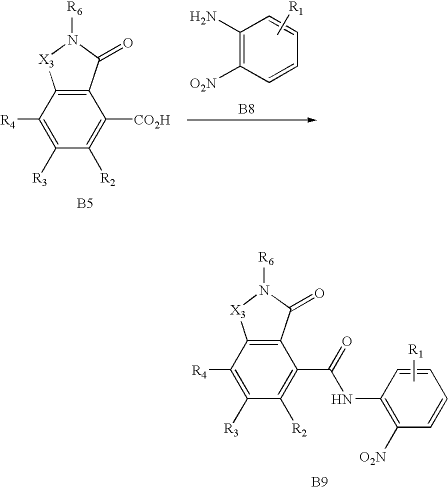

Alternatively, Compound B5 is coupled with a suitable amino nitro Compound B8 using a coupling reagent (such as EDCI in a suitable solvent such as DMF) to provide a Compound B9.

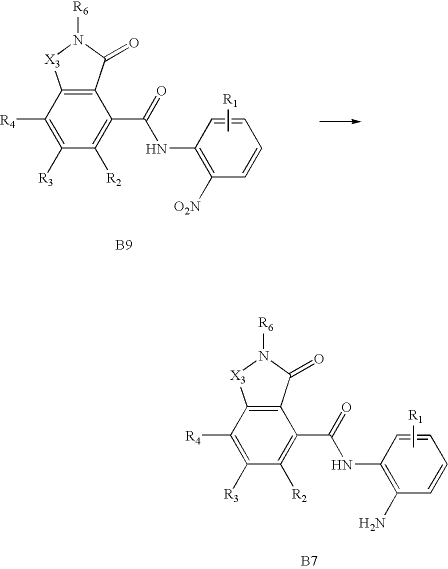

Compound B9 is reduced with a suitable reagent such as SnCl₂ in a solvent (such as DMF or hydrogen) and a suitable catalyst (such as Pd on charcoal in a suitable solvent such as EtOH) to afford Compound B7.

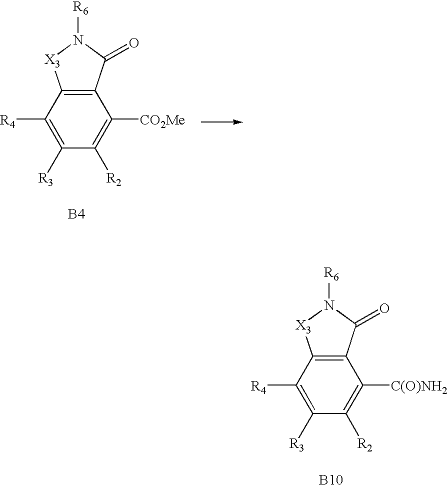

Compound B4 is treated with ammonium hydroxide or ammonia in a suitable solvent such as THF to provide a Compound B10.

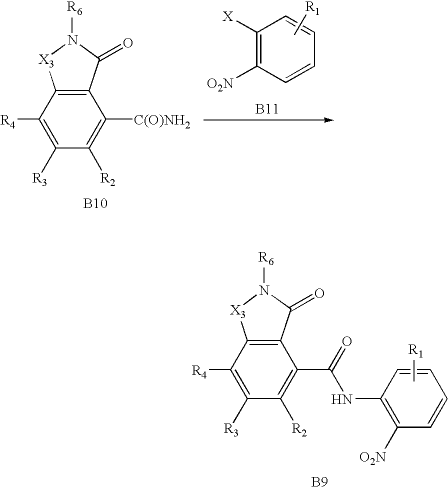

Compound B10 is reacted with a nitro Compound B11 (wherein X is F, Cl, Br, I or SO₃CF₃) and a base (such as Cs₂CO₃ or (i-Pr)₂NEt in a suitable solvent such as DMF) to afford Compound B9.

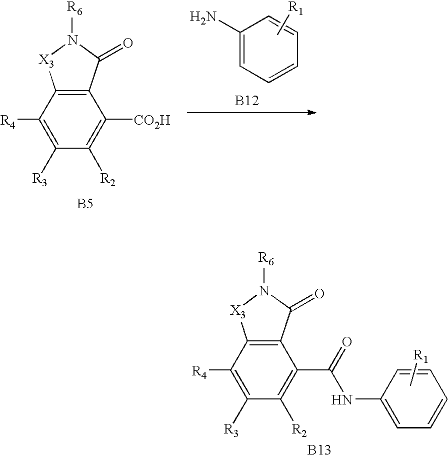

Alternatively, Compound B5 is coupled with a suitable amino Compound B12 using a coupling reagent (such as EDCI in a suitable solvent such as DMF) to provide a Compound B13.

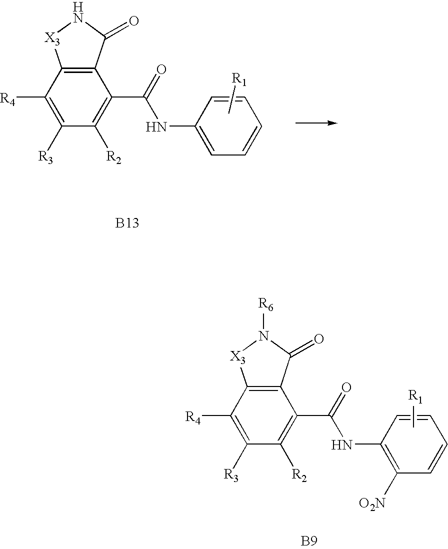

Compound B13 is nitrated with a suitable agent (such as HNO₃) to provide Compound B9.

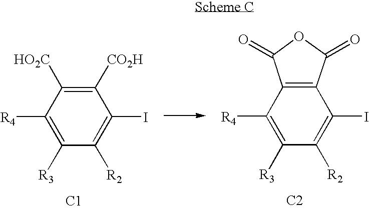

Compound C1 is cyclized in a suitable solvent such as refluxing acetic anhydride to provide a Compound C2.

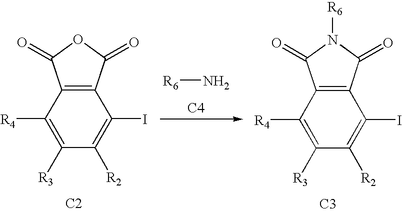

Compound C2 is treated with a suitable aminating reagent such as urea or Compound C4 to provide a Compound C3.

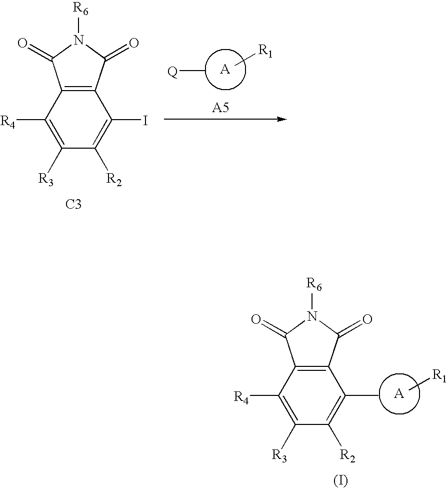

Compound C3 is reacted with a Compound A5 (wherein Q is a boronic acid or ester and the like) in the presence of a palladium catalyst (such as Pd(OAc)₂, Pd(dppf)₂Cl₂, Pd(PPh₃)₄ and the like) to provide compound of formula (I), wherein X₃ is —C(O)—.

Alternatively, Compound C3 is reacted with a boronating reagent (such as boronic acid or ester such as bis(pinacolato)diboron and the like) to form a boronated intermediate amenable for further reaction with a bromine substituted Compound A5 (wherein Q is bromine and the like) to also provide a compound of formula (I), wherein X₃ is —C(O)—.

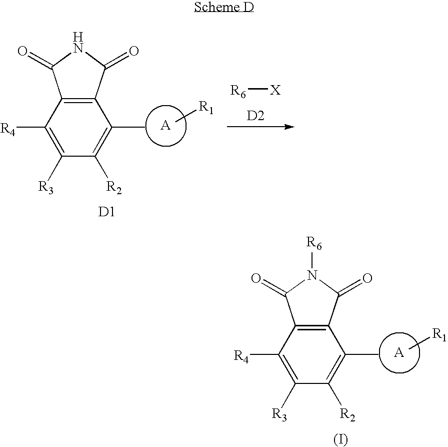

Compound D1 is treated with a Compound D2 (wherein X is Cl, Br, I, SO₃C₆H₄CH₃, SO₃CH₃ or SO₃CF₃) and suitable a base (such as NaH, t-BuOK or Et₃N in a solvent such as THF or DMF) to provide a compound of formula (I), wherein X₃ is —C(O)—.

Alternatively, the compound of formula (I), wherein X₃ is —C(O)— is treated with a reducing agent (such as NaBH₄ or Et₃SiH and TFA) in a suitable solvent (such as CH₂Cl₂ or EtOH) to provide a compound of the formula (I), wherein X₃ is CH₂.

### SPECIFIC SYNTHETIC EXAMPLES

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The depicted intermediates may also be used in subsequent examples to produce additional compounds of the present invention. No attempt has been made to optimize the yields obtained in any of the reactions. One skilled in the art would know how to increase such yields through routine variations in reaction times, temperatures, solvents and/or reagents.

General: 1H and C¹³ NMR spectra were obtained at 400 MHz and 300 MHz on a Brucker AVANCE300 and AVANCE400 spectrometer. Chemical shifts are reported in ppm downfield from TMS as an internal standard. Magnesium sulfate was employed to dry organic extracts prior to concentration by rotary evaporation. Flash chromatography was done using EM science silica gel 60 (230-400 mesh).

Standard solvents from J. T. Baker were used as received. Anhydrous solvents from Aldrich or J.T.Baker and all other commercially available reagents were used without further purification.

Silica gel (E. Merck, 230-400 mesh) was used for all flash chromatography. Thin-layer chromatography was performed on precoated plates with silica gel 60 F254 from EM Science. Yields were not optimized.

Mass electrospray positive or negative spectra (MS) was performed on Hewlett Packard 1100 series or Agilent 1100 series spectrometer with a Zorbax stablebond C¹⁸ narrow bore column, using gradient 0.05% acetic acid in MeOH and 0.05% acetic acid in water as mobile phase for MS analysis, and using gradient 0.05% TFA in acetonitrile and 0.05% acetic acid in water as mobile phase for LCMS analysis.

HPLC quantitative purity analysis were additionally carried on Agilent 1100 Series LC/MSD equipment on a Agilent 4.6×50 mm Zorbax 3.5 uM column (Elips XDB-phenyl) using gradient 0.05% TFA acetonitrile and 0.05% TFA in water as solvent system and based on the absorption at 254 nM.

### Example 1

### 5-benzyloxy-2-(3-oxo-2,3-dihydro-1H-isoindol-4-yl)-indole-1-carboxylic acid tert-butyl ester (Compound 32)

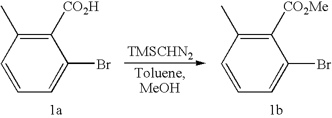

A solution of 2-bromo-6-methyl-benzoic acid Compound 1a (15.5 g, 72 mmol) in toluene/methanol (3/1, 360 mL) was treated with TMSCHN₂ (2M in Hexanes, 40 mL, 80 mmol) and stirred at 25° C. for 1 hour. The reaction mixture was concentrated and the residue purified by column chromatography (SiO₂, 0-25% EtOAc/Hex) to yield 2-bromo-6-methyl-benzoic acid methyl ester Compound 1b (14.5 g, 88%) as an orange oil. ¹H NMR (300 MHz, CDCl₃) δ 7.40 (m, 1H), 7.15 (d, J=4.8 Hz, 2H), 3.96 (s, 3H), 2.34 (s, 3H); MS (ESI) m/z: did not hit (MH⁺).

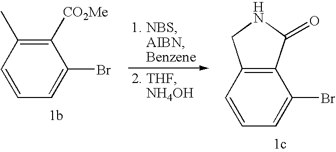

A solution of Compound 1b (65.9 g, 288 mmol), NBS (53.8 g, 302 mmol) and AIBN (2.3 g, 14 mmol) in benzene (1 L) was warmed at reflux for 8 hours. The reaction mixture was cooled to 25° C. and the succinimide filtered off and washed with hexanes. The filtrate was concentrated, dissolved in THF (1 L), treated with conc NH₄OH (290 mL) and stirred at 25° C. for 4 hours. The reaction was diluted with H₂O (1 L), and extracted with EtOAc (3×300 mL). The organic extracts were concentrated and purified by column chromatography (SiO₂) to yield 7-bromo-2,3-dihydro-isoindol-1-one Compound 1c (28.9 g, 47%) as an off-white solid. ¹H NMR (400 MHz, CDCl₃) δ 7.73 (br s, 1H), 7.62 (d, J=7.2 Hz, 1H), 7.43 (m, 2H), 4.42 (s, 2H); MS (ESI) m/z: did not hit (MH⁺).

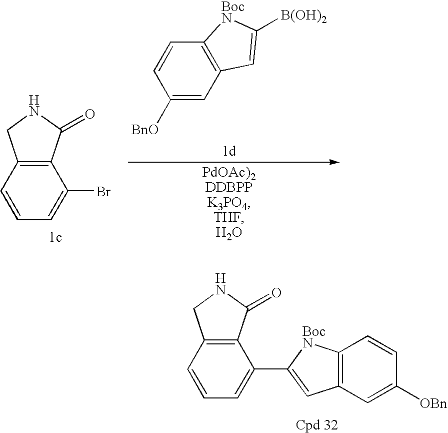

A suspension of 2-borono-5-benzyloxy-indole-1-carboxylic acid tert-butyl ester Compound 1d (31.2 g, 84.9 mmol), Compound 1c (12.0 g, 56.6 mmol), Pd(OAc)₂ (254 mg, 1.13 mmol), DDBPP (464 mg, 1.13 mmol), K₃PO₄ (freshly powdered, 36.0 g, 170 mmol) and H₂O (3.05 mL, 170 mmol) was stirred with an overhead mechanical stirrer for 24 hours at 25° C. The reaction mixture was diluted with H₂O (1 L) and extracted with EtOAc/THF (3/1, 3×300 mL). The organic extracts were dried with Na₂SO₄ and concentrated. The residue was purified by column chromatography (SiO₂, 5-100% THF/Hex) to yield 5-benzyloxy-2-(3-oxo-2,3-dihydro-1H-isoindol-4-yl)-indole-1-carboxylic acid tert-butyl ester Compound 32 (23.2 g, 90%) as an off white solid. ¹H NMR (300 MHz, CDCl₃) δ 8.17 (d, J=9 Hz, 1H), 7.62-7.03 (m, 11H), 6.51 (s, 1H), 5.15 (s, 2H), 4.44 (s, 2H), 1.29 (s, 9H); MS (ESI) m/z: 477 (MNa⁺).

Using the procedure of Example 1, other compounds of the present invention were prepared:

| Cpd | Name | MS |
|---|---|---|
| 15 | 5-methoxy-2-(7-nitro-3-oxo-2,3-dihydro-1H-isoindol- | 424 |
| | 4-yl)-indole-1-carboxylic acid tert-butyl ester | |
| 26 | 2-(3-oxo-2,3-dihydro-1H-isoindol-4-yl)-indole-1- | 349 |
| | carboxylic acid tert-butyl ester | |
| 27 | 5-methoxy-2-(3-oxo-2,3-dihydro-1H-isoindol-4-yl)- | 379 |
| | indole-1-carboxylic acid tert-butyl ester | |
| 31 | 5-Methoxy-2-(6-methoxy-3-oxo-2,3-dihydro-1H-isoindol- | 409 |
| | 4-yl)-indole-1-carboxylic acid tert-butyl ester | |

### Example 2

### 5-hydroxy-2-(3-oxo-2,3-dihydro-1H-isoindol-4-yl)-indole-1-carboxylic acid tert-butyl ester (Compound 28)

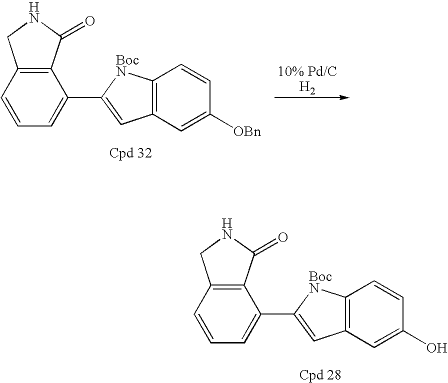

A solution of Compound 32 (1.0 g, 2.2 mmol) in THF/MeOH (3/1, 22 mL) was treated with 10% Pd/C (100 mg) and stirred at 25° C. under H₂ (balloon) for 3 hours. The reaction mixture was then filtered through a pad of Celite and the filtrate concentrated to yield 5-hydroxy-2-(3-oxo-2,3-dihydro-1H-isoindol-4-yl)-indole-1-carboxylic acid tert-butyl ester Compound 28 (0.84 g, 100%) as an off-white solid. ¹H NMR (300 MHz, DMSO-d6) δ 9.12 (s, 1H), 8.58 (s, 1H), 7.94 (d, J=8.7 Hz, 1H), 7.60 (m, 2H), 7.40 (d, J=6 Hz, 1H), 6.91 (d, J=2.4 Hz, 1H), 6.80 (dd, J=8.7, 2.4 Hz, 1H), 6.46 (s, 1H), 4.38 (s, 2H), 1.12 (s, 9H); MS (ESI) m/z: 387 (MNa⁺).

### Example 3

### 2-(3-oxo-2,3-dihydro-1H-isoindol-4-yl)-5-(3-piperidin-1-yl-ethoxy)-indole-1-carboxylic acid tert-butyl ester (Compound 29)

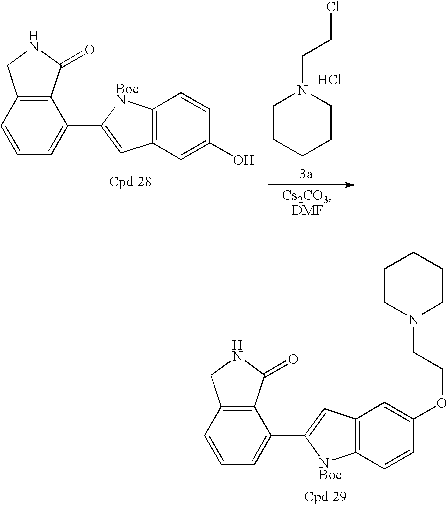

A suspension of Compound 28 (5.0 g, 13.7 mmol), Cs₂CO₃ (13.4 g, 41 mmol), and 1-(2-chloro-ethyl)-piperidine monohydrochloride Compound 3a (2.5 g, 13.7 mmol) in DMF (69 mL) was warmed at 50° C. for 24 hours. The reaction mixture was diluted with H₂O (300 mL) and extracted with EtOAc/THF (3/1, 3×300 mL). The extracts were concentrated and purified by column chromatography (SiO₂, O-3% 2M N₃ in MeOH/DCM) to yield 2-(3-oxo-2,3-dihydro-1H-isoindol-4-yl)-5-(2-piperidin-1-yl-ethoxy)-indole-1-carboxylic acid tert-butyl ester Compound 29 (5.3 g, 81%) as an off-white solid. ¹H NMR (400 MHz, DMSO-d6) δ 8.60 (s, 1H), 8.04 (d, J=8.8 Hz, 1H), 7.63 (m, 2H), 7.43 (d, J=6.0 Hz, 1H), 7.15 (d, J=2.8 Hz, 1H), 6.94 (dd, J=9.2, 2.8 Hz, 1H), 6.53 (s, 1H), 4.40 (s, 2H), 4.10 (t, J=6.4 Hz, 2H), 2.69 (t, J=5.6 Hz, 2H), 2.49 (m, 4H), 1.53 (m, 4H), 1.40 (m, 2H), 1.14 (s, 9H); MS (ESI) m/z: 476 (MH⁺).

Using the procedure of Example 3, other compounds of the present invention were prepared:

| Cpd | Name | MS |
|---|---|---|
| 30 | 5-(2-morpholin-4-yl-ethoxy)-2-(3-oxo-2,3-dihydro-1H- | 478 |
| | isoindol-4-yl)-indole-1-carboxylic acid tert-butyl ester | |

### Example 4

### 7-[5-(2-piperidin-1-yl-ethoxy)-1H-indol-2-yl]-2,3-dihydro-isoindol-1-one (Compound 10)

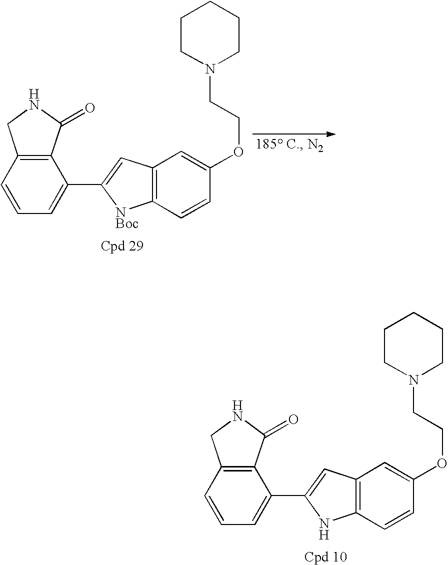

Compound 29 (5.2 g, 10.9 mmol) was warmed at 185° C. under N₂ for 1 hour in the absence of solvent. The residue was purified by column chromatography (SiO₂, 0-5% 2M N₃ in MeOH/DCM) to yield Compound 10 (2.4 g, 59%) as a pale yellow solid. ¹H NMR (300 MHz, DMSO-d6) δ 13.83 (s, 1H), 9.30 (s, 1H), 8.15 (d, J=7.8 Hz, 1H), 7.66 (t, J=7.8 Hz, 1H), 7.49 (d, J=7.2 Hz, 1H), 7.38 (d, J=8.7 Hz, 1H), 7.14 (s, 1H), 7.08 (d, J=2.1 Hz, 1H), 6.78 (dd, J=9.0, 2.4 Hz, 1H), 4.52 (s, 2H), 4.08 (t, J=5.7 Hz, 2H), 2.68 (t, J=6.3 Hz, 2), 2.47 (m, 4H), 1.53 (m, 4H), 1.40 (m, 2H); MS (ESI) m/z: 377 (MH⁺).

Using the procedure of Example 4, other compounds of the present invention were prepared:

| Cpd | Name | MS |
|---|---|---|
| 1 | 7-{5-[3-(4-ethyl-piperazin-1-yl)-propoxy]-1H-indol-2-yl}-2,3-dihydro- | 419 |
| | isoindol-1-one | |
| 2 | 7-{5-[3-(4-hydroxymethyl-piperidin-1-yl)-propoxy]-1H-indol-2-yl}-2,3- | 420 |
| | dihydro-isoindol-1-one | |
| 3 | 7-(5-methoxy-1H-indol-2-yl)-2,3-dihydro-isoindol-1-one | 279 |
| 5 | 7-[5-(3-hydroxy-propoxy)-1H-indol-2-yl]-2,3-dihydro-isoindol-1-one | 323 |
| 6 | 5-methoxy-7-(5-methoxy-1H-indol-2-yl)-2,3-dihydro-isoindol-1-one | 309 |
| 7 | 7-(1H-indol-2-yl)-2,3-dihydro-isoindol-1-one | 249 |
| 8 | 7-[5-(2-morpholin-4-yl-ethoxy)-1H-indol-2-yl]-2,3-dihydro-isoindol-1-one | 378 |
| 9 | 7-[5-(3-piperidin-1-yl-propoxy)-1H-indol-2-yl]-2,3-dihydro-isoindol-1-one | 390 |

### Example 5

### 5-benzyloxy-2-(6-hydroxy-3-oxo-2,3-dihydro-1H-isoindol-4-yl)-indole-1-carboxylic acid tert-butyl ester (Compound 13)

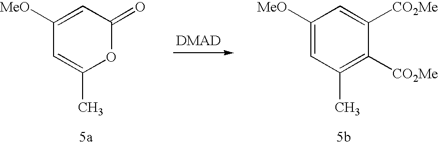

4-methoxy-6-methyl-pyran-2-one Compound 5a (25.3 g, 180.5 mmol) in DMAD (neat, 33.3 mL, 271.0 mmol) was heated in a round bottom flask at 180° C. for 1 hr and then at 210° C. for 2 hrs. The reaction mixture was cooled to room temperature and concentrated onto SiO₂ (75 g), then purified via column chromatography (Horizon, 65% to 60% gradient starting with 5% EtOAc/Hexanes to 20% EtOAc/Hexanes) to afford 5-methoxy-3-methyl-phthalic acid dimethyl ester Compound 5b (30.12 g, 70%) as a colorless oil. ¹H NMR (300 MHz, CDCl₃) δ 7.29, (d, J=2.1 Hz, 1H), 6.91 (d, 2.0 Hz, 1H), 3.99 (s, 3H), 3.90 (s, 3H), 3.84 (s, 3H), 234 (s, 3H).

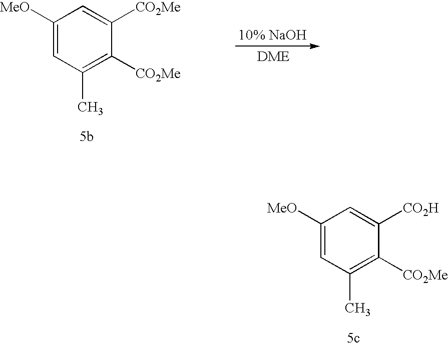

5-methoxy-3-methyl-phthalic acid dimethyl ester Compound 5b (30.0 g, 126 mmol) was stirred in DME (168 ml, 0.75 M) at 25° C., then 10% NaOH (168 ml) was added. After TLC analysis (30% EtOAc/Hexanes; 1N HCl/EtOAc) showed that the reaction was complete, the reaction mixture was diluted with EtOAc (250 ml) and transferred to a separatory funnel. The organic layer was washed with 1N HCl (3×150 ml), then dried (Na₂SO₄) and concentrated to afford 5-methoxy-3-methyl-phthalic acid 2-methyl ester Compound 5c (25.9 g, 92%) as a white solid. ¹H NMR (400 MHz, CDCl₃) δ 7.40 (d, J=2.8 Hz, 1H), 6.97 (d, J=2.4 Hz, 1H), 3.91 (s, 3H), 3.87 (s, 3H), 2.36 (s, 3H).

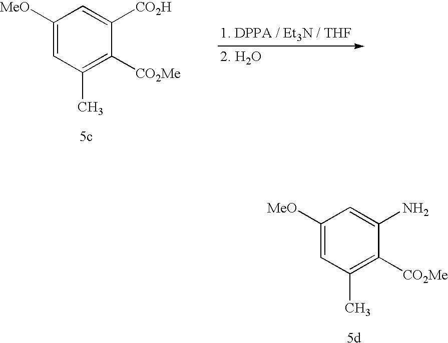

5-methoxy-3-methyl-phthalic acid 2-methyl ester Compound 5c (25.9 g, 115.5 mmol) was stirred in THF (500 ml, 0.23M) 25° C., then TEA (24.1 ml, 173.0 mmol) and DPPA (26.2 ml, 121.3 mmol) were added. The reaction mixture continued to stir at 25° C. After TLC analysis (50% EtOAc/Hexanes) showed that the reaction was complete, H₂O was added (188 ml) and the reaction mixture was heated to reflux temperature for 3 hrs. TLC analysis (30% EtOAc/Hexanes; H₂O/EtOAc) showed the absence of the isocyanate intermediate. The reaction mixture was cooled to room temperature and concentrated onto SiO₂ (50 g), then purified via column chromatography (Horizon, 65% to 60% gradient starting with 100% Hexanes to 20% EtOAc/Hexanes) to afford 2-amino-4-methoxy-6-methyl-benzoic acid methyl ester Compound 5d (18.56 g, 82%). ¹H NMR (300 MHz, CDCl₃) δ 6.14 (d, J=2.1 Hz, 1H), 6.02 (d, J=2.0 Hz, 1H), 5.48 (br s, 2H), 3.87 (s, 3H), 3.82, (s, 3H), 2.44 (s, 3H).

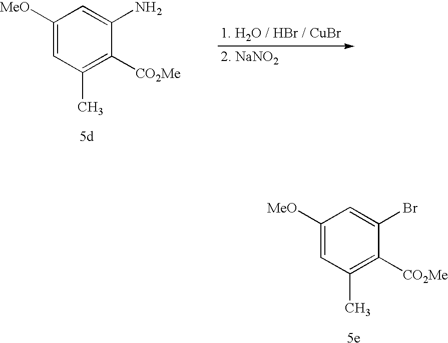

A mixture of 2-amino-4-methoxy-6-methyl-benzoic acid methyl ester Compound 5d (18.6 g, 95.3 mmol), 48% HBr (32.4 ml, 286.0 mmol) and H₂O (238 ml) was heated to 90° C. Copper bromide (45.1 g, 314 mmol) was added in portions and then a solution of sodium nitrite in H₂O was added drop wise. The mixture was then stirred at 90° C. for 0.5 hrs. The mixture was poured into a beaker containing ice, diluted with EtOAc (300 ml) and THF (300 ml), then filtered through a bed of Celite. The filtrate was transferred to a separatory funnel, the organic layer was removed and concentrated onto SiO₂ (70 g), then purified via column chromatography (Horizon, 65% to 60% gradient starting with 100% Hexanes to 5% EtOAc/Hexanes) to afford 2-bromo-4-methoxy-6-methyl-benzoic acid methyl ester Compound 5e (9.98 g, 40%) as a colorless oil. ¹H NMR (400 MHz, CDCl3) δ 6.96 (d, J=2.4 Hz, 1H), 6.69 (d, J=2.3 Hz, 1H), 3.86 (3, H), 3.81 (s, 3H), 2.33 (s, 3H).

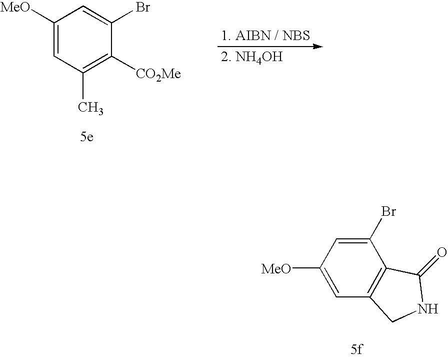

A solution of 2-bromo-4-methoxy-6-methyl-benzoic acid methyl ester Compound 5e (1.17 g, 2.74 mmol), NBS (7.2 g, 40.5 mmol) and AIBN (0.31 g, 1.9 mmol) in benzene (150 ml, 0.24 M) was heated to reflux temperature for 5 hrs. NMR analysis showed a 72% conversion to Compound 5f along with 27% of Compound 5e. The reaction mixture was diluted with H₂O (150 ml), filtered through a bed of Celite and concentrated to a viscous red oil, which was used in the next step without any further purification. The red oil intermediate was diluted with THF (400 ml) at 25° C., then concentrated ammonium hydroxide (50 ml) was added. The reaction mixture was stirred for 12 hrs and then transferred to a separatory funnel. The organic layer was washed with H₂O (400 ml) and concentrated onto SiO₂ (25 g), then purified via column chromatography (Horizon, 65% to 60% gradient starting with 70% EtOAc/Hexanes to 100% EtOAc) to afford 7-bromo-5-methoxy-2,3-dihydro-isoindol-1-one Compound 5f (3.7 g, 40%). ¹H NMR (400 MHz, CDCl3) δ 7.20 (s, 1H), 7.17 (d, 2.0 Hz, 1H), 6.93 (d, J=1.2 Hz, 1H), 4.39 (s, 2H), 3.89 (s, 3H).

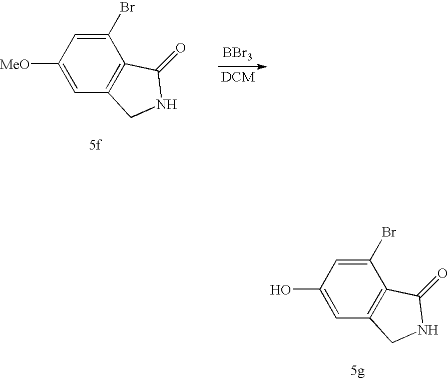

A solution of 7-bromo-5-methoxy-2,3-dihydro-isoindol-1-one Compound 5f (1.0 g, 4.13 mmol) in DCM (20 ml, 02.M) was stirred at 25° C., then 1M BBr₃ (6.2 ml, 6.2 mmol) was added. The mixture was stirred at 25° C. for 3 hrs while additional BBr₃ (4.0 ml) was added, then heated to reflux for 3 hrs. The reaction mixture was poured into a beaker containing ice, then diluted with EtOAc (100 ml) and extracted with EtOAc (2×75 ml). The organic layer was washed with brine (2×50 ml), then dried (Na₂SO₄) and concentrated to afford 7-bromo-5-hydroxy-2,3-dihydro-isoindol-1-one Compound 5g (0.65 g, 69%). ¹H NMR (300 MHz, DMSO-d6) δ 10.50, (s, 1H), 8.35, (s, 1H), 7.00 (d, J=1.8 Hz, 1H), 6.90 (d, J=0.9H, 1H), 4.22 (s, 2H).

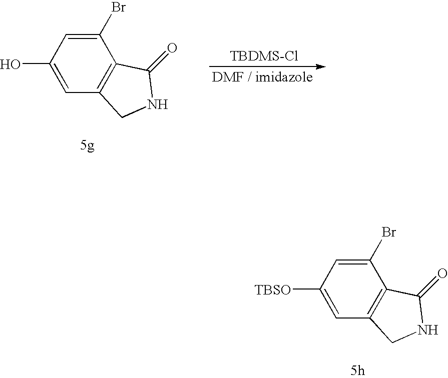

A solution of 7-bromo-5-hydroxy-2,3-dihydro-isoindol-1-one Compound 5g (0.605 g, 2.65 mmol), imidazole (0.45 g, 6.6 mmol) and TBDMS-Cl (0.48 g, 3,18 mmol) in DMF (27 ml, 01.M) was stirred at 25° C. for 6 hrs. Additional TBDMS-Cl (0.2 g, 1.3 mmol) was added and the reaction mixture was stirred at 25° C. for an additional 4 hrs. After TLC analysis (70% EtOAc/Hexanes) showed that the reaction was complete, H₂O (30 ml) was added. Sat'd. NaHCO₃ (30 ml) was added to the reaction mixture, which was then extracted with EtOAc (3×75 ml). The organic layer was dried (Na₂SO₄) and concentrated under reduced pressure to afford 7-bromo-5-(tert-butyl-dimethyl-silanyloxy)-2,3-dihydro-isoindol-1-one Compound 5h (0.788 g, 87%) as a white solid. ¹H NMR (400 MHz, CDCl3) δ 7.10, (d, J=2.0 Hz, 1), 6.85 (br s, 1H), 6.85 (d, J=1.2 Hz, 1H), 4.34 (s, 2H), 1.00, (s, 9H), 0.27, (s, 6H).

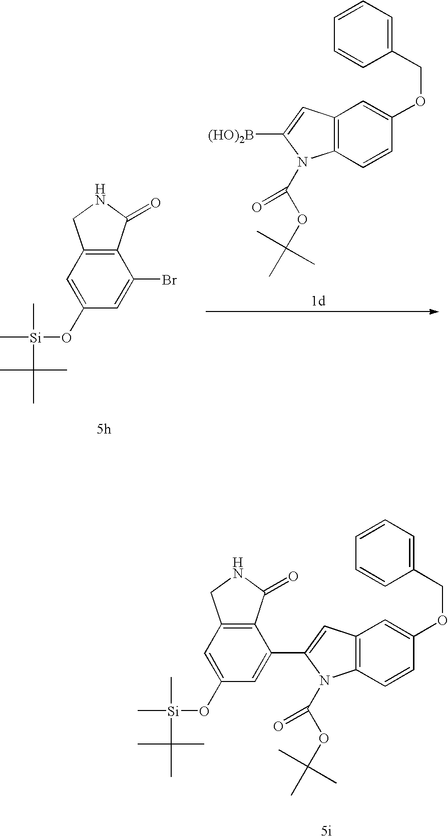

Using the procedure of Example 1 and Compound 5h in place of Compound 1c, additional compounds of the present invention were prepared such as 5-benzyloxy-2-[6-(tert-butyl-dimethyl-silanyloxy)-3-oxo-2,3-dihydro-1H-isoindol-4-yl]-indole-1-carboxylic acid tert-butyl ester Compound 5i.

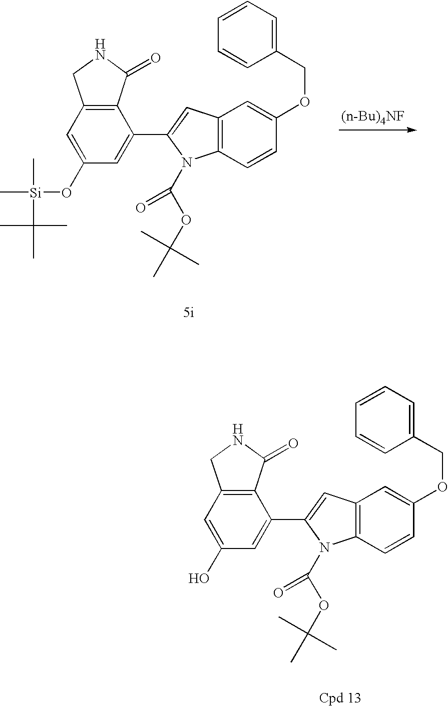

Compound 5i was treated with (n-Bu)₄NF (TBAF) in THF at room temperature to provide, after extractive workup and purification by column chromatography using ethyl acetate:hexanes, Compound 13 as an off-white solid. ¹H NMR (300 MHz, CDCl₃) 1.32 (s, 9H), 4.28 (broad s, 2H), 5.07 (s, 2H), 6.47 (s, 1H), 6.81 (m, 2H), 6.90 (m, 1H), 7.01 (m, 2H), 7.25-7.5 (complex, 6H), 8.06 (d, J-8.9 Hz, 1H); MS m/z 471 (MH⁺).

### Example 6

### 5-methoxy-2-(7-nitro-3-oxo-2,3-dihydro-1H-isoindol-4-yl)-indole-1-carboxylic acid tert-butyl ester (Compound 15)

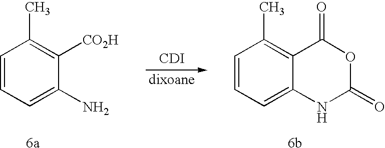

As described in Bioorg. & Med. Chem. Lett., 1997, Vol 7 (16), 2105-2108, commercially available 2-amino-6-methyl-benzoic acid Compound 6a was used to prepare 5-methyl-1H-benzo[d][1,3]oxazine-2,4-dione Compound 6b.

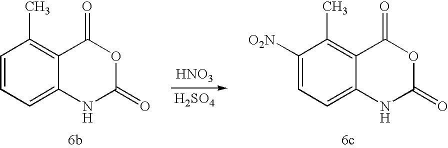

As described in WO 2004/063198, Compound 6b was carried forward to provide 5-methyl-6-nitro-1H-benzo[d][1,3]oxazine-2,4-dione Compound 6c.

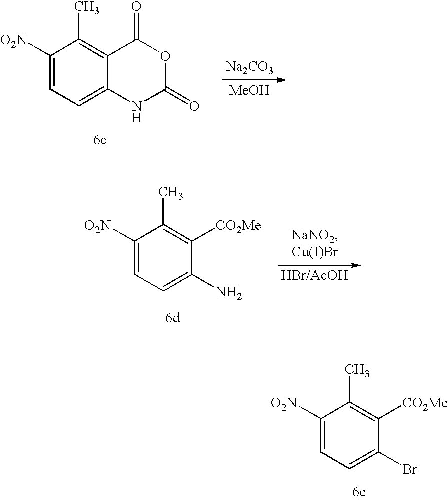

As further described in WO 2004/063198, Compound 6c was used to prepare 6-amino-2-methyl-3-nitro-benzoic acid methyl ester Compound 6d and to subsequently provide 6-bromo-2-methyl-3-nitro-benzoic acid methyl ester Compound 6e.

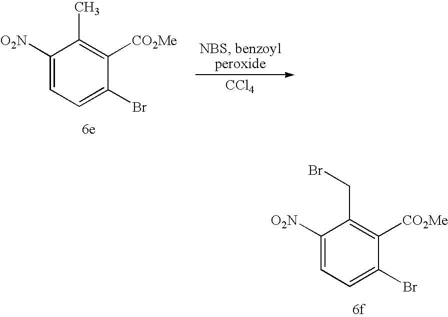

A solution of 6-bromo-2-methyl-3-nitro-benzoic acid methyl ester Compound 6e (6.4 g, 23.35 mmol; commercially available) in carbon tetrachloride (200 mL, 0.11M) was stirred at 25° C., then NBS (6.23 g, 35.03 mmol) and benzoyl peroxide (0.85 g, 3.50 mmol) were added. The reaction mixture was stirred at reflux temperature for 24 hrs under a nitrogen atmosphere. NBS (6.23 g, 35.05 mmol) and benzoyl peroxide (0.85 g, 3.50 mmol) were added and the mixture was stirred for 48 hrs. After NMR (DMSO) analysis showed that the reaction was substantially complete (77% conversion to product), the reaction mixture was concentrated onto SiO₂ (18 g), then purified via column chromatography (Horizon, 65% to 60% gradient starting with 15% DCM/Hexanes to 35% DCM/Hexanes) to afford 6-bromo-2-bromomethyl-3-nitro-benzoic acid methyl ester Compound 6f (6.34 g, 77%) as a yellow solid. ¹H NMR (300 MHz, DMSO-d6) δ 8.1-8.02, (dd, J=3.0 Hz, 2H), 4.67, (s, 2H), 3.98, (s, 3H).

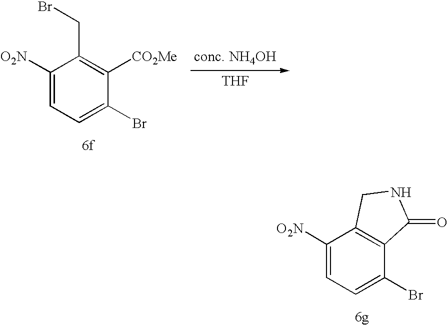

A solution of 6-bromo-2-bromomethyl-3-nitro-benzoic acid methyl ester Compound 6f (6.09 g, 17.25 mmol) in THF was stirred at 25° C., then concentrated ammonium hydroxide (15 ml) was added. After TLC analysis (25% EtOAc/Hexanes) showed that the reaction was complete, the THF was removed under reduced pressure and H₂O (50 ml) was added. A white precipitate was filtered off and the solids were dried under reduced pressure. The solids were triturated (10:1 Hexanes:EtOAc) for 3 hrs, then filtered off to provide 7-bromo-4-nitro-2,3-dihydro-isoindol-1-one Compound 6g. ¹H NMR (300 MHz, DMSO-d6) δ 9.14 (br s, 1H), 8.30 (d, J=8.4 Hz, 2H), 7.97 (d, J=8.7 Hz, 2H), 4.74 (s, 2H).

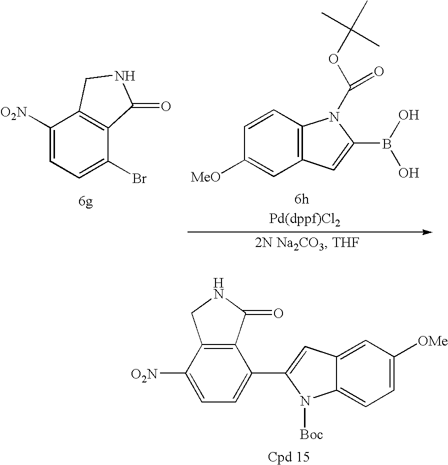

A round bottom flask was charged with 7-bromo-4-nitro-2,3-dihydro-isoindol-1-one Compound 6g (0.86 g, 3.34 mmol), 1-boc-5-methoxyindole-2-boronic acid Compound 6h (1.17 g, 4.0 mmol) and Pd(dppf)Cl₂ (0.27 g, 0.334 mmol) in 2N Na₂CO₃ (8 ml) and THF (16 ml). The reaction mixture was heated to reflux temperature for 2 hrs. The aqueous layer was separated and concentrated down, the organic layer was concentrated onto SiO₂ (3 g), then purified via column chromatography (Horizon, 65% to 60% gradient starting with 2% EtOAc/Hexanes to 20% EtOAc/Hexanes) to afford 5-methoxy-2-(7-nitro-3-oxo-2,3-dihydro-1H-isoindol-4-yl)-indole-1-carboxylic acid tert-butyl ester Compound 15 (1.17 g, 82%) as a yellow solid. ¹H NMR (400 MHz, CDCl3) δ 8.46, (d, J=8.4 Hz, 1H), 8.08, (d, J=12.0 Hz, 1H), 7.68, (d, J=8.4 Hz, 1H), 7.04-6.99, (m, 2H), 6.62, (2, 2H), 4.91, (br s, 2H), 3.86, (s, 3H), 1.42, (s, 9H); MS m/z 424 (MH⁺).

Using the procedure of Example 6, other compounds of the present invention were prepared:

| Cpd | Name | MS |
|---|---|---|
| 33 | 5-benzyloxy-2-(7-nitro-3-oxo-2,3-dihydro-1H-isoindol-4- | 500 |
| | yl)-indole-1-carboxylic acid tert-butyl ester | |

### Example 7

### 7-(5-methoxy-1H-indol-2-yl)-4-nitro-2,3-dihydro-isoindol-1-one (Compound 16)

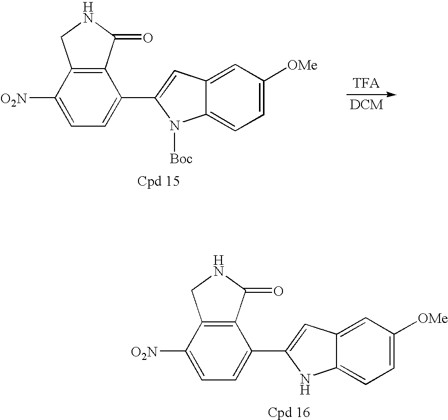

A solution of 5-methoxy-2-(7-nitro-3-oxo-2,3-dihydro-1H-isoindol-4-yl)-indole-1-carboxylic acid tert-butyl ester Compound 15 (1.17 g, 2.74 mmol) in DCM (15 ml, 0.18 M) was stirred at room temperature, then TFA (15 ml) was added. After TLC analysis (2:3 EtOAc/Hexanes) showed that the reaction was complete, the DCM/TFA was removed under reduced pressure. The solids were diluted with DCM and washed with sat. NaHCO₃ (3×30 ml), then dried (Na₂SO₄) and concentrated to afford 7-(5-methoxy-1H-indol-2-yl)-4-nitro-2,3-dihydro-isoindol-1-one Compound 16 (0.88 g, 98%) as a red colored solid. ¹H NMR (400 MHz, DMSO-d6) δ 13.99, (s, 1H), 9.70, (s, 1H), 8.39, (s, 2H), 7.45-7.41, (m, 2H), 7.07, (s, 1H), 6.91-6.85, (m, 1H), 4.90, (s, 2H), 3.78, (s, 3H).

### Example 8

### 4-amino-7-(5-methoxy-1H-indol-2-yl)-2,3-dihydro-isoindol-1-one (Compound 17)

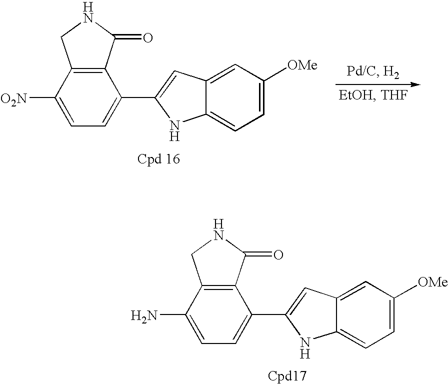

A solution of 7-(5-methoxy-1H-indol-2-yl)-4-nitro-2,3-dihydro-isoindol-1-one Compound 16 (0.88 g, 2.72 mmol) and 10% Pd/C (10 mg, 10% wt/wt) in EtOH (15 ml, 0.18M) were stirred for 24 hrs at room temperature under a H₂ atmosphere. The homogenous dark solution was filtered through a bed of Celite and the filtrate was concentrated under reduced pressure, then purified via column chromatography (Horizon, 65% to 60% gradient starting with 100% DCM to 10% MeOH/DCM) to afford 4-amino-7-(5-methoxy-1H-indol-2-yl)-2,3-dihydro-isoindol-1-one Compound 17 (0.29 g, 37%) as a green solid. ¹H NMR (300 MHz, DMSO-d6) δ 12.79, (br s, 1H), 8.92, (br s, 1H), 7.36, (d, J=8.4 Hz, 1H), 7.27, (d, J=8.7 Hz, 1H), 6.64, (dd, J=10.8 Hz, 1H), 6.39-6.31, (m, 2H), 5.42, (br s, 2H), 4.17, (br s, 2H), 3.34, (s, 3H).

### Example 9

### N-[7-(5-methoxy-1H-indol-2-yl)-1-oxo-2,3-dihydro-1H-isoindol-4-yl]-acetamide (Compound 18)

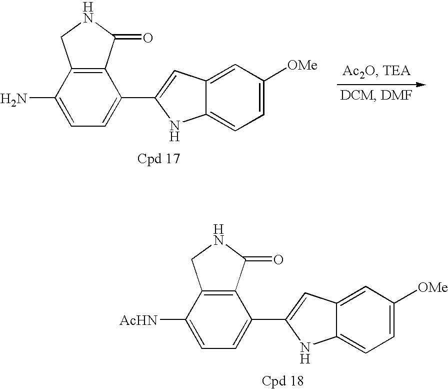

A solution of 4-amino-7-(5-methoxy-1H-indol-2-yl)-2,3-dihydro-isoindol-1-one Compound 17 (0.05 g, 0.174 mmol), Ac₂O (18 uL, 0.19 mmol) and TEA (30 uL, 0.21 mmol) were stirred under a nitrogen atmosphere in DCM (1 ml) and DMF (1 ml). The reaction mixture was heated to 40° C. for 12 hrs, then concentrated onto SiO₂ (0.3 g) and purified via column chromatography (Horizon, 65% to 60% gradient starting with 100% DCM to 2% MeOH/DCM) to afford N-[7-(5-methoxy-1H-indol-2-yl)-1-oxo-2,3-dihydro-1H-isoindol-4-yl]-acetamide Compound 18 (0.036 g, 65%) as a white solid. ¹H NMR (400 MHz, DMSO-d6) δ 12.61, (br s, 1H), 9.59, (br s, 1H), 8.98, (s, 1H), 7.50-7.48, (m, 2H), 7.32, (d, J=8.8 Hz, 1H), 6.69, (d, J=11.6 Hz, 1H), 6.39, (s, 1H), 4.37-4.36, (m, 2H), 3.40, (s, 3H), 2.04, (s, 3H).

### Example 10

### 7-(5-benzyloxy-1H-indol-2-yl)-2,3-dihydro-isoindol-1-one (Compound 19)

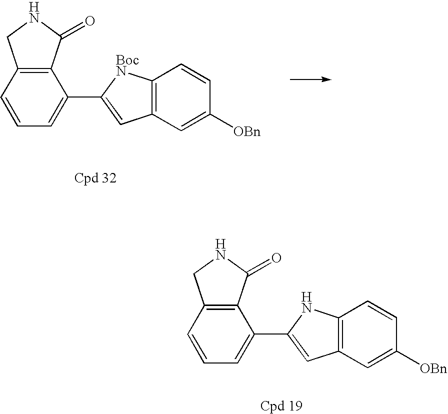

Compound 1e was warmed at 185° C. under N₂ for 1 hour in the absence of solvent. The residue was purified by column chromatography to yield Compound 19 as a tan solid. ¹H NMR (300 MHz, DMSO-d6) 4.50 (s, 2H), 5.12 (s, 2H), 6.85 (dd, J=2.4, 8.7 Hz, 1H), 7.14 (d, J=2.4 Hz, 1H), 7.3-7.5 (complex, 6H), 7.62 (t, J=7.8 Hz, 1H), 8.14 (d, J=7.8 Hz, 1H), 9.28 (s, 1H), 13.8 (broad s, 1H); MS (ESI) m/z: 355 (MH⁺).

### Example 11

### 7-(5-hydroxy-1H-indol-2-yl)-2,3-dihydro-isoindol-1-one (Compound 4)

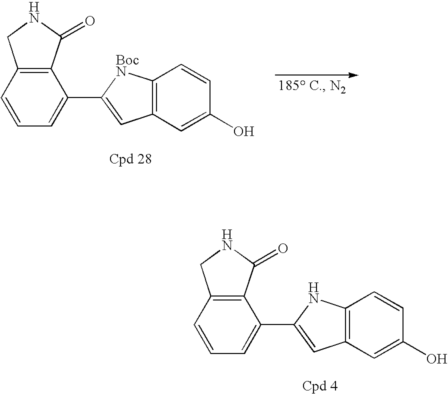

Compound 28 was warmed at 185° C. under N₂ for 1 hour in the absence of solvent. The residue was purified by column chromatography to yield Compound 4 as an off-white solid. ¹H NMR (400 MHz, MeOD) 4.49 (s, 2H), 6.7 (dd, J=2.3, 8.7 Hz, 1H), 6.83 (m, 2H), 7.23 (d, J=8.3 Hz, 1H), 7.38 (d, J=7.7 Hz, 1H), 7.57 (t, J=7.7 Hz, 1H), 8.2 (d, J=7.7 Hz, 1H), 13.3 (broad s, 1H); MS (ESI) m/z: 265 (MH⁺).

Using the procedure of Example 8, other compounds of the present invention were prepared:

| Cpd | Name | MS |
|---|---|---|
| 14 | 7-(5-benzyloxy-1H-indol-2-yl)-5-hydroxy-2,3- | 371 |
| | dihydro-isoindol-1-one | |

### Example 12

### 2-(3-oxo-2,3-dihydro-1H-isoindol-4-yl)-pyrrole-1-carboxylic acid tert-butyl ester (Compound 23)

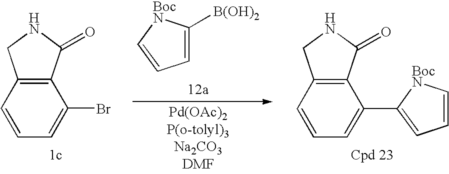

Similar to the procedure used to prepare Compound 32, Compound 1c was reacted with Compound 12a and Pd(OAc)₂, P(o-tolyl)₃, and 2N aqueous Na₂CO₃ in DMF to give Compound 23 as a clear, colorless oil. ¹H NMR (400 MHz, CDCl₃) 1.30 and 1.65 (rotamer A and B, two singlets, 9H), 4.42 (s, 2H), 6.25 (m, 1H), 7.35-7.65 (complex, 5H); MS (ESI) m/z: 299 (MH⁺).

### Example 13

### 7-(1H-pyrrol-2-yl)-2,3-dihydro-isoindol-1-one (Compound 12)

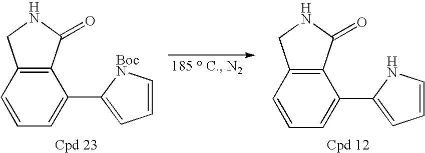

Compound 23 was warmed at 185° C. under N₂ for 1 hour in the absence of solvent. The crude product was purified by column chromatography to provide Compound 12 as an off-white solid. ¹H NMR (400 MHz, CDCl₃) 4.49 (s, 2H), 6.29 (m, 1H), 6.47 (broad s, 1H), 6.83 (m, 1H), 6.98 (m, 1H), 7.16 (d, J=7.5 Hz, 1H), 7.49 (t, J=7.8 Hz, 1H), 7.86 (d, J=8.2 Hz, 1H), 13.45 (broad s, 1H); MS (ESI) m/z: 199 (MH⁺).

### Example 14

### 2-(2-methyl-3-oxo-2,3-dihydro-1H-isoindol-4-yl)-pyrrole-1-carboxylic acid tert-butyl ester (Compound 24)

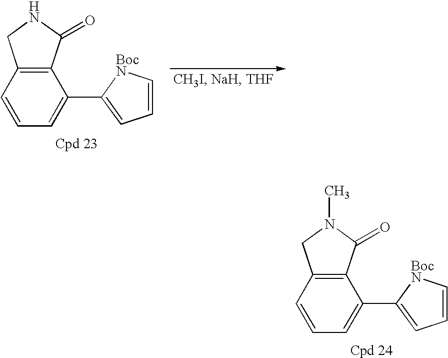

Compound 23 was dissolved in THF and added to a stirring suspension of sodium hydride (NaH) in THF. Methyl iodide was added and the mixture was stirred under N₂. Compound 24 was isolated after workup. MS (ESI) m/z: 313 (MH⁺).

### Example 15

### 2-methyl-7-(1H-pyrrol-2-yl)-2,3-dihydro-isoindol-1-one (Compound 25)

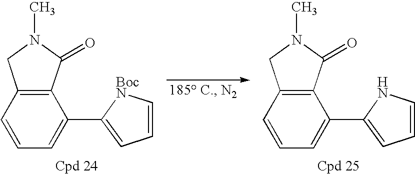

Compound 24 was warmed at 185° C. under N₂ for 1 hour in the absence of solvent. The crude material was purified by column chromatography to provide Compound 25 as an off-white solid. ¹H NMR (400 MHz, CDCl₃) 3.22 (s, 3H), 4.41 (s, 2H), 6.28 (m, 1H)6.81 (m, 1H), 6.96 (m, 1H), 7.12 (dd, J=0.8, 7.2 Hz, 1H), 7.44 (t, J=7.5 Hz, 1H), 7.83 (d, J=7.7 Hz, 1H), 13.7 (broad s, 1H); MS (ESI) m/z: 213 (MH⁺).

### Example 16

### 7-(5-methoxy-1H-benzoimidazol-2-yl)-2,3-dihydro-isoindol-1-one (Compound 11)

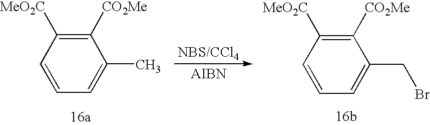

3-methyl-phthalic acid dimethyl ester Compound 16a was treated with N-bromosuccinimide (NBS) in carbon tetrachloride in the presence of AIBN to provide 3-bromomethyl-phthalic acid dimethyl ester Compound 16b.

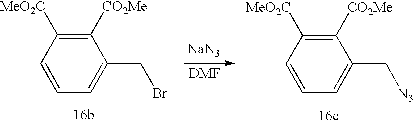

Compound 16b was reacted with sodium azide in DMF to give 3-azidomethyl-phthalic acid dimethyl ester Compound 16c.

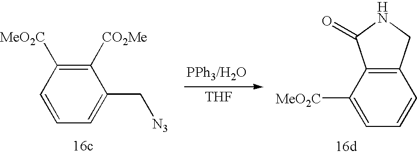

Compound 16c was in turn converted to 3-oxo-2,3-dihydro-1H-isoindole-4-carboxylic acid methyl ester Compound 16d in an overall yield of 52% (3 steps). ¹H NMR (300 MHz, CDCl₃) 4.01 (s, 3H), 4.47 (s, 2H), 7.55-7.75 (complex, 3H); MS (ESI) m/z: 192 (MH⁺).)

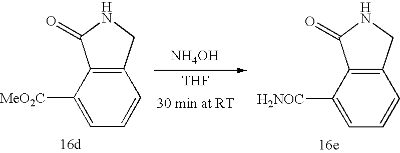

Compound 16d was dissolved in THF and treated with concentrated ammonium hydroxide at room temperature for 30 min to give 3-oxo-2,3-dihydro-1H-isoindole-4-carboxylic acid amide 16e ¹H NMR (300 MHz, DMSO-d₆) 4.64 (s, 2H), 7.84 (t, J=7.6 Hz, 1H), 7.92 (d, J=7 Hz, 1H), 8.17 (d, J=7.7 Hz, 1H), 10.15 (broad s, 1H); MS (ESI) m/z: 177 (MH⁺).

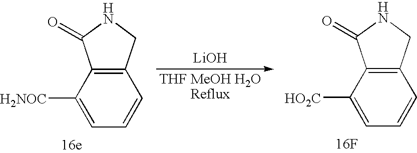

Compound 16e was treated with lithium hydroxide in a refluxing mixture of THF, MeOH, and water to provide 3-oxo-2,3-dihydro-1H-isoindole-4-carboxylic acid Compound 16f. ¹H NMR (300 MHz, MeOD) 4.51 (s, 2H), 7.75 (m, 2H), 8.32 (m, 1H); MS (ESI) m/z: 178 (MH⁺).

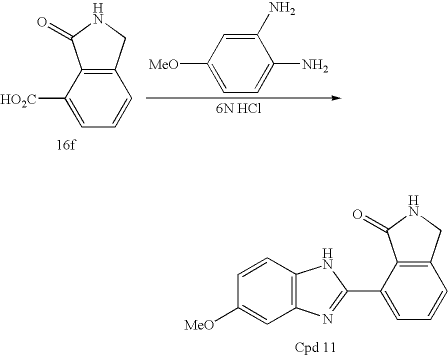

Compound 16f was treated with 4-methoxy-benzene-1,2-diamine in 6N HCl to provide Compound 11. ¹H NMR (400 MHz, MeOD) 3.87 (s, 3H), 4.56 (s, 2H), 6.92 (d, J=8.7 Hz, 1H), 7.15 (s, 1H), 7.55 (broad s, 1H), 7.65 (d, J=7.6 Hz, 1H), 7.75 (t, J=7.6 Hz, 1H), 8.56 (d, J=7.6 Hz, 1H); MS (ESI) m/z: 280 (MH⁺).

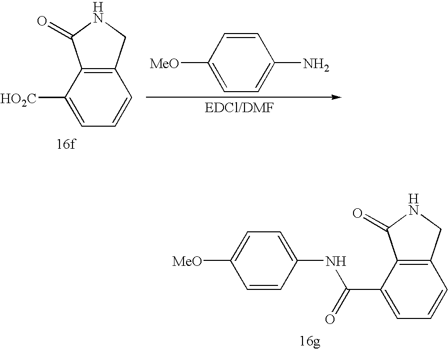

Compound 16f was coupled with 4-methoxyaniline using a coupling agent such as EDCI in DMF to give 3-oxo-2,3-dihydro-1H-isoindole-4-carboxylic acid (4-methoxy-phenyl)-amide Compound 16g ¹H NMR (300 MHz, MeOD) 3.80 (s, 3H), 4.56 (s, 2H), 6.94 (d, J=9 Hz, 2H), 7.70 (d, J=9 Hz, 2H), 7.78 (m, 2H), 8.40 (m, 1H); MS (ESI) m/z: 283 (MH⁺).

### Example 17

### 2-(1,3-dioxo-2,3-dihydro-1H-isoindol-4-yl)-5-methoxy-indole-1-carboxylic acid tert-butyl ester (Compound 21)

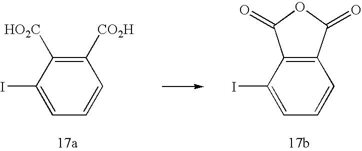

Commercially available 3-iodo-1,2-benzendicarboxylic acid Compound 17a was cyclized to 4-iodophthalic anhydride Compound 17b in refluxing acetic anhydride. ¹H NMR (300 MHz, CDCl₃) 7.57 (t, J=7.7 Hz, 1H), 8.01 (d, J=7.6 Hz, 1H), 8.30 (d, J=7.7 Hz, 1H); MS (ESI) m/z: 275 (MH⁺).

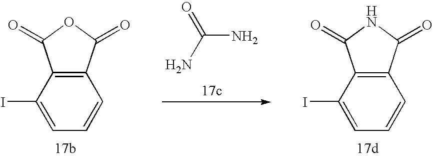

Compound 17b was converted to 4-iodophthalimide Compound 17d by treatment with urea Compound 17c. MS (ESI) m/z: 274 (MH⁺).

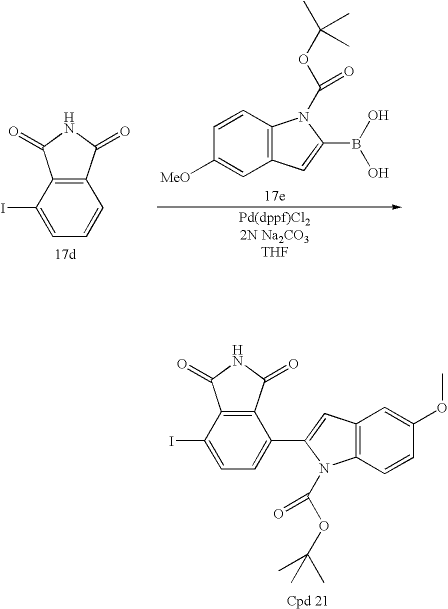

Compound 17d was coupled with commercially available 1-(t-butoxycarbonyl)-5-methoxyindole-2-boronic acid Compound 17e using Pd(dppf)Cl₂ and 2N aqueous Na₂CO₃ in THF to afford Compound 21. ¹H NMR (300 MHz, CDCl₃) 1.39 (s, 9H), 3.87 (s, 3H), 6.60 (s, 1H), 6.99 (dd, J=2.5, 9.2 Hz, 1H), 7.04 (d, J=2.5 Hz, 1H), 7.68 (broad s, 1H), 7.7-7.8 (complex, 2H), 7.87 (dd, J=1.6, 7.0 Hz, 1H), 8.09 (d, J=9.1 Hz, 1H); MS (ESI) m/z: 393 (MH⁺).

### Example 18

### 4-(5-methoxy-1H-indol-2-yl)-isoindole-1,3-dione (Compound 22)

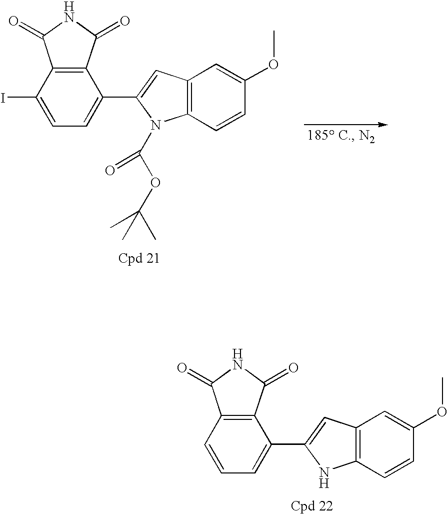

Compound 21 was warmed at 185° C. under N₂ in the absence of solvent until starting material was completely consumed. The residue was purified by SiO₂ chromatography to provide Compound 22 as a red solid. ¹H NMR (300 MHz, DMSO-d₆) 3.87 (s, 3H), 6.84 (dd, J=2.4, 9.1 Hz, 1H), 7.10 (d, J=2.2 Hz, 1H), 7.29 (d, J=1.5 Hz, 1H), 7.44 (d, J=8.9 Hz, 1H), 7.74 (d, J=7.3 Hz, 1H), 7.86 (t, J=7.6 Hz, 1H), 8.32 (d, J=8 Hz, 1H), 11.68 (s, 1H), 11.98 (s, 1H); MS (ESI) m/z: 293 (MH⁺).

### Example 19

### 7-(1H-Indol-3-yl)-2,3-dihydro-isoindol-1-one (Compound 20)

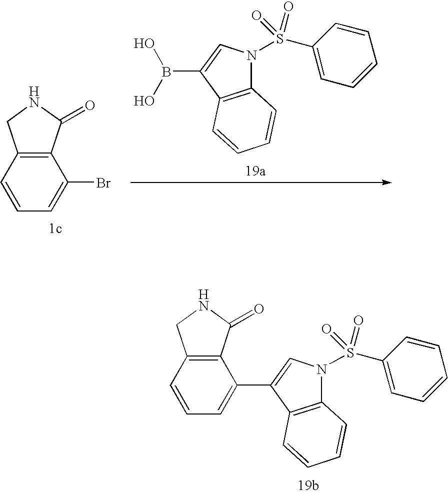

Using the procedure of Example 1 and Compound 19a in place of Compound 1d, Compound 1c was converted to 7-(1-benzenesulfonyl-1H-indol-3-yl)-2,3-dihydro-isoindol-1-one Compound 19b, which was isolate as a white solid. ¹H NMR (300 MHz, MeOD) 4.48 (s, 2H), 7.22 (dt, J=1, 8 Hz, 1H), 7.31 (dt, J=1, 8 Hz, 1H), 7.45-7.75 (complex, 7H), 7.45-8.1 (complex, 4H); MS (ESI) m/z: 389 (MH⁺).

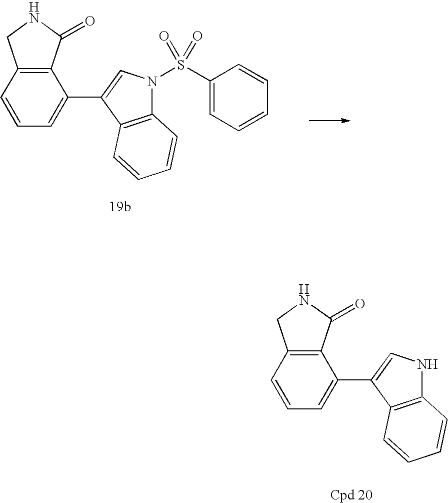

Compound 19b was treated with aqueous NaOH in dioxane to afford Compound 20 as a yellow solid. ¹H NMR (300 MHz, MeOD) 4.44 (s, 2H), 7.05 (dt, J=1, 8 Hz, 1H), 7.12 (dt, J=1, 8 Hz, 1H), 7.35-7.75 (complex, 6H); MS (ESI) m/z: 249 (MH⁺).

### BIOLOGICAL EXAMPLES

The ability of the compounds to treat or ameliorate protein kinase mediated disorders was determined using the following procedures.

### Example 1

VEGF-R2 and Aurora-A Screening Assays

A kinase reaction mixture was prepared containing 50 mM Tris-HCl pH=8, 10 mM MgCl₂, 0.1 mM Na₃PO₄, 1 mM DTT, 10 μM ATP, 0.025 μM biotinylated histone-H1 peptide substrate and 0.2 μCuries per well ^{33P}-γ-ATP (2000-3000 Ci/mmol). 70 μL of the kinase reaction mixture was dispensed into the well of a streptavidin coated FlashPlate™ (Cat. # SMP103, NEN, Boston, Mass.).

Test compound stock in 100% DMSO (1 μL) was added to the wells resulting in a final concentration of 1% DMSO in the reaction with a 100 μL final reaction volume. Each enzyme was diluted in 50 mM Tris-HCl pH=8.0, 0.1% BSA and 30 μL was added to each well to initiate the reaction. The reaction was incubated for one hour at 30° C. At the end of the 1 hr incubation, the reaction was terminated by aspirating the mixture from the plate and washing the wells twice with PBS containing 100 mM EDTA. The biotinylated peptide substrate became immobilized on the Flashplate™ and the incorporation of ³³P-γ-ATP was measured by reading the plate on a scintillation counter. Inhibition of the enzymatic activity was measured by observing a reduced amount of ³³P-γ-ATP incorporated into the immobilized peptide.

The VEGF-R2 enzyme is a fusion protein containing a polyhistidine tag at the N terminus followed by amino acids 786 to 1343 of the rat VEGF-R2 kinase domain (Accession number U93306). The assay used 150 ng of the N-terminal biotinylated peptide biotin-KHKKLAEGSAYEEV-amide (VEGF-R2) per well.

Aurora-A is a fusion protein containing a polyhistidine tag at the N terminus followed by the full length protein encoding the murine Aurora-A (Accession number GB BC014711) expressed and purified from sf9 insect cells. The assay used 400 ng of the N-terminal biotinylated peptide biotin-GRTGRRNSI-amide (Aurora-A) per well.

A maximum and minimum signal for the assay was determined on each plate. The percent inhibition of a test compound was calculated according to the formula:

$$[ \frac{( max signal - test compound )}{( max signal - min signal )} ] ( 100 ) = \% inhibition$$

For a series of test concentrations, the IC₅₀ was derived by graphing percent inhibition against the log of the concentrations tested for a given compound with results shown in Table 1.

For those compounds without an IC₅₀, the inhibition values in percent are shown at a single test concentration of 0.1, 10 or 100 μM, as noted.

TABLE 1
Kinase IC₅₀ (μM)

| Cpd | VEGF-R2 | Aurora-A |
|---|---|---|
| 1 | 0.014 | 0.044 |
| 2 | 0.018 | 0.061 |
| 3 | 0.111 | 0.070 |
| 4 | 0.119 | 0.090 |
| 5 | 0.030 | 0.100 |
| 6 | 0.103 | 0.100 |
| 7 | 0.144 | 0.100 |
| 8 | 0.074 | 0.135 |
| 9 | 0.065 | 0.179 |
| 10 | 0.056 | 0.200 |
| 11 | 0.142 | 0.205 |
| 12 | 1.095 | 0.338 |
| 15 | 1.52 | 0.270 |
| 16 | 0.0229 | 52% @ 10 μM |
| 17 | 0.0532 | 0.432 |
| 18 | >100 | 64% @ 100 μM |
| 19 | 11.3 | 21.2 |
| 20 | 50% inh @ 100 μM | 60% inh @ 100 μM |
| 21 | 1.52 | 0.270 |
| 22 | 35% inh @ 0.1 μM | 30% inh @ 0.1 μM |
| 23 | 46% inh @ 100 μM | 25% inh @ 100 μM |
| 25 | 37% inh @ 100 μM | >100 |
| 26 | 34% @ 100 μM | 61% @ 100 μM |
| 27 | 57% @ 100 μM | 48% @ 10 μM |
| 28 | 62% @ 100 μM | 9.35 |
| 29 | 63% @ 100 μM | 58% @ 100 μM |
| 30 | 65% @ 100 μM | 57% @ 100 μM |
| 31 | 17% @ 100 μM | 40% @ 100 μM |

### Example 4

Cell Proliferation Inhibition Assay

The ability of a test compound to inhibit unregulated cell proliferation may be determined by measuring incorporation of ¹⁴C-labelled thymidine into newly synthesized DNA within cell lines derived from carcinomas originating from several tissues. Accordingly, the effect of a test compound on proliferation of cells with a variety of phenotypes may be determined.

Carcinoma cell lines used include the HeLa cervical adenocarcinoma from the American Type Culture Collection (ATCC Cat. #CCL2), A375 malignant melanoma (ATCC Cat. #CRL-1619) and HCT-116 colon carcinoma (ATCC Cat. #CCL-247).

The carcinoma cells are trypsinized and counted. The cells (3000-8000 count) are added to each well of a 96-well CytoStar tissue culture treated scintillating microplate (Amersham #RPNQO160) in complete medium (100 μL) and the plate is then incubated in complete medium for 24 hrs at 37° C. in an inert atmosphere containing 5% CO₂.

Test compound (1 μL) in 100% DMSO is added to the plate test-wells with DMSO only added to control-wells. The plate is incubated in complete medium for a second 24 hr period at 37° C. in an atmosphere containing 5% CO₂.

An aliquot of a solution of Methyl ¹⁴C-thymidine (56 mC/mmol) (NEN #NEC568 or Amersham #CFA532) and complete medium (20 uL to provide 0.2 μCi/well) is then added to each well and the plate is incubated for a third 24 hr period at 37° C. in an atmosphere containing 5% CO₂.

The plate contents are then discarded, the plate is washed twice with PBS (200 μL) and then PBS (200 μL) is added to each well. The plate is sealed and the degree of methyl ¹⁴C-thymidine incorporation is quantified on a Packard Top Count.

For a series of test concentrations, the IC₅₀ was derived by graphing percent inhibition against the log of the concentrations tested for a given compound with results shown in Table 2.

TABLE 2
Cell Proliferation IC₅₀ (μM)

| Cpd | HeLa | A375 | HCT116 |
|---|---|---|---|
| 1 | 5.31 | 3.90 | 3.85 |
| 2 | 3.35 | 0.763 | 2.26 |
| 3 | >100 | >100 | >100 |
| 4 | 3.64 | 4.85 | 6.09 |
| 5 | 15 | 4.40 | 6.47 |
| 6 | 5.08 | 3.02 | 4.28 |
| 7 | 3.71 | 5.56 | 5.04 |
| 8 | 5.81 | 2.70 | 5.74 |
| 9 | 6.06 | 4.03 | 7.77 |
| 10 | 4.63 | 0.437 | 2.34 |
| 11 | >10 | >10 | >10 |
| 12 | 52.6 | >10 | >100 |
| 16 | 9.42 | 5.23 | 3.09 |
| 21 | 43.0 | >100 | >100 |
| 27 | 55.9 | >10 | >10 |
| 28 | 34.8 | 34.4 | >10 |
| 29 | 14.6 | 4.14 | >1 |
| 30 | 20.3 | 5.31 | 17.0 |
| 31 | 44.3 | 39.6 | 39.5 |

### Example 5

In Vivo Models—Inhibition of Tumor Growth

The ability of test compounds to inhibit unregulated growth of human tumor cells in vivo was evaluated by implanting human tumor cells into the hindflank of athymic mice, administering a test compound and then quantifying any change in tumor size.

Human epidermoid A431 carcinoma and N87 cells are implanted subcutaneously into the hindflank of female athymic mice (Charles River) and allowed to grow for 6-10 days. After a measurable tumor is established (as determined by baseline caliper measurement), the animal was administered an oral dose of the test compound daily for a period of 30 days. Tumor size is measured every five days and the degree of inhibition is determined by comparing drug-treated animals to vehicle-treated animals.

Variations of this method are intended to include intraperitoneal injection or intravenous infusion as the route of administration and administration of the test compound either alone or in a combination therapy.

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. The disclosure of these publications is hereby incorporated by reference into this application to describe more fully the state of the art to which this invention pertains.

What is claimed is:

1. A compound of formula (I): 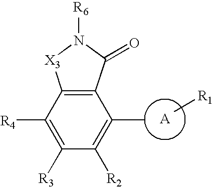
or a form thereof, wherein ring A is a heteroaromatic monocyclic or bicyclic ring system moiety;
X₃ is selected from the group consisting of CH₂ and C═O;
R₁ is one, two, three, four or five substituents each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₂, R₃ and R₄ is each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₅ is one, two, three, four or five substituents each selected from the group consisting of hydrogen, halogen, hydroxy, nitro, cyano, C₁₋₈alkyl, C₁₋₈alkoxy, C₁₋₈alkyl-halogen, C₁₋₈alkoxy-halogen, C₁₋₈alkyl-hydroxy, C₁₋₈alkoxy-hydroxy, amino, amino-C₁₋₈alkyl, C₁₋₈alkyl-amino and C₁₋₈alkyl-amino-C₁₋₈alkyl; and
R₆ is selected from the group consisting of hydrogen, optionally substituted C₁₋₈alkyl, C₃₋₈cycloalkyl-R₅, aryl-R₅, heteroaryl-R₅ and heterocyclyl-R₅,
wherein C₁₋₈alkyl is optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅.

2. A compound of formula (Ia): 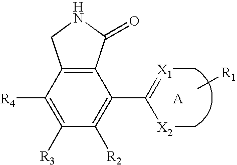
or a form thereof, wherein
Ring A is taken together with X₁ and X₂ to form a heteroaromatic monocyclic or bicyclic A(X₁,X₂) ring system moiety;
X₁ is selected from the group consisting of N and CH;
X₂ is selected from the group consisting of NH, CH and CH₂; wherein X₁ and X₂ cannot simultaneously be CH and CH₂;
R₁ is one, two, three, four or five substituents each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy;
R₂, R₃ and R₄ is each selected from the group consisting of hydrogen, halogen, hydroxy, nitro, cyano, optionally substituted C₁₋₈alkyl, optionally substituted C₁₋₈alkoxy, optionally substituted amino, optionally substituted carbamoyl, carbonyl-C₁₋₈alkoxy, C₃₋₈cycloalkyl-R₅, aryl-R₅, heteroaryl-R₅ and heterocyclyl-R₅,
wherein C₁₋₈alkyl and C₁₋₈alkoxy is each optionally substituted with one, two, three, four or five substituents each selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino, C₁₋₈alkyl-amino-C₁₋₈alkyl, C₁₋₈alkyl-C₁₋₈alkoxy, C₁₋₈alkyl-C₃₋₈cycloalkyl-R₅, C₁₋₈alkyl-aryl-R₅, C₁₋₈alkyl-heteroaryl-R₅, C₁₋₈alkyl-heterocyclyl-R₅, C₁₋₈acyl, C₁₋₈acyl-amino-C₁₋₈alkyl, C₁₋₈acyl-C₃₋₈cycloalkyl-R₅, C₁₋₈acyl-aryl-R₅, C₁₋₈acyl-heteroaryl-R₅, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino, C₁₋₈alkyl-amino-C₁₋₈alkyl and C₁₋₈alkyl-C₁₋₈alkoxy; and
R₅ is one, two, three, four or five substituents each selected from the group consisting of hydrogen, halogen, hydroxy, nitro, cyano, C₁₋₈alkyl, C₁₋₈alkoxy, C₁₋₈alkyl-halogen, C₁₋₈alkoxy-halogen, C₁₋₈alkyl-hydroxy, C₁₋₈alkoxy-hydroxy, amino, amino-C₁₋₈alkyl, C₁₋₈alkyl-amino and C₁₋₈alkyl-amino-C₁₋₈alkyl.

3. The compound of claim 2, wherein the A(X₁,X₂) ring system moiety is selected from the group consisting of pyrrol-2-yl, imidazol-2-yl, pyrazol-2-yl, indol-2-yl, isoindol-1-yl and benzimidazol-2-yl.

4. The compound of claim 2, wherein the A(X₁,X₂) ring system moiety is selected from the group consisting of pyrrol-2-yl, indol-2-yl and benzimidazol-2-yl.

5. The compound of claim 2, wherein R₁ is one, two, three, four or five substituents each selected from the group consisting of hydrogen, halogen, hydroxy, optionally substituted C₁₋₈alkyl, optionally substituted C₁₋₈alkoxy, optionally substituted amino, optionally substituted carbamoyl and carbonyl-C₁₋₈alkoxy,
wherein C₁₋₈alkyl and C₁₋₈alkoxy is each optionally substituted with one substituent selected from the group consisting of C₁₋₈alkoxy, halogen, hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₁₈alkyl-amino-C₁₋₈alkyl, C₁₋₁₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₁₈alkyl-amino, C₁₋₁₈alkyl-amino-C₁₋₈alkyl and C₁₋₁₈alkyl-C₁₋₈alkoxy.

6. The compound of claim 2, wherein R₁ is one or two substituents selected from the group consisting of hydrogen, halogen, hydroxy, optionally substituted C₁₋₈alkoxy, optionally substituted amino, optionally substituted carbamoyl and carbonyl-C₁₋₈alkoxy,
wherein C₁₋₈alkoxy is optionally substituted with one substituent selected from the group consisting of hydroxy, amino, amino-C₁₋₈alkyl, amino-C₁₋₈alkyl-C₁₋₈alkoxy, aryl-R₅, and heterocyclyl-R₅,
wherein amino is optionally substituted with one or two substituents each selected from the group consisting of C₁₋₈alkyl, C₁₋₈alkyl-amino-C₁₋₈alkyl, C₁₋₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl-amino-C₁₋₈alkyl and C₁₋₈alkyl-C₁₋₈alkoxy.

7. The compound of claim 2, wherein R₁ is one or two substituents selected from the group consisting of hydrogen, hydroxy, optionally substituted C₁₋₈alkoxy, and carbonyl-C₁₋₈alkoxy; wherein C₁₋₈alkoxy is optionally substituted with one substituent selected from the group consisting of hydroxy, aryl-R₅, and heterocyclyl-R₅.

8. The compound of claim 2, wherein R₂, R₃ and R₄ is each selected from the group consisting of hydrogen, halogen, hydroxy, nitro, optionally substituted C₁₋₈alkyl, optionally substituted C₁₋₈alkoxy, optionally substituted amino, optionally substituted carbamoyl and carbonyl-C₁₋₈alkoxy,
wherein C₁₋₈alkyl and C₁₋₈alkoxy is each optionally substituted with one, two, three, four or five substituents each selected from the group consisting of hydroxy, amino, amino-C₁₋₈alkyl, amino-C₁₋₈alkyl-C₁₋₈alkoxy, C₃₋₈cycloalkyl-R₅, aryl-R₅, heteroaryl-R₅ and heterocyclyl-R₅,
wherein amino is optionally substituted with one or two substituents each selected from the group consisting of C₁₋₈alkyl, C₁₋₈alkyl-amino-C₁₋₈alkyl, C₁₋₈alkyl-C₁₋₈alkoxy, C₁₋₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl, C₁₋₈alkyl-amino-C₁₋₈alkyl and C₁₋₈alkyl-C₁₋₈alkoxy.

9. The compound of claim 2, wherein R₂, R₃ and R₄ is each selected from the group consisting of hydrogen, halogen, hydroxy, nitro, C₁₋₈alkyl, optionally substituted C₁₋₈alkoxy, optionally substituted amino, optionally substituted carbamoyl and carbonyl-C₁₋₈alkoxy,
wherein C₁₋₈alkyl and C₁₋₈alkoxy is each optionally substituted with one, two, three, four or five substituents each selected from the group consisting of hydroxy, amino, amino-C₁₋₈alkyl and amino-C₁₋₈alkyl-C₁₋₈alkoxy,
wherein amino is optionally substituted with one or two substituents each selected from the group consisting of C₁₋₈alkyl-amino-C₁₋₈alkyl, C₁₋₁₈alkyl-C₁₋₈alkoxy, C₁₋₁₈alkyl-heterocyclyl-R₅, C₁₋₈acyl, C₁₋₈acyl-amino-C₁₋₈alkyl, C₁₋₈acyl-heterocyclyl-R₅, aroyl-R₅, heteroaroyl-R₅ and heterocycloyl-R₅, and
wherein carbamoyl is optionally substituted on amino with one or two substituents each selected from the group consisting of C₁₋₈alkyl and C₁₋₈alkyl-amino-C₁₋₈alkyl.

10. The compound of claim 2, wherein R₂, R₃ and R₄ is each selected from the group consisting of hydrogen, halogen, hydroxy, nitro, C₁₋₈alkyl, optionally substituted C₁₋₈alkoxy, optionally substituted amino, and carbonyl-C₁₋₈alkoxy,
wherein C₁₋₈alkoxy is optionally substituted with one substituent selected from the group consisting of hydroxy, amino, amino-C₁₋₈alkyl and amino-C₁₋₈alkyl-C₁₋₈alkoxy; and
wherein amino is optionally substituted with one substituent selected from the group consisting of C₁₋₈alkyl, C₁₋₈alkyl-amino-C₁₋₈alkyl, C₁₋₈acyl, and C₁₋₈acyl-amino-C₁₋₈alkyl.

11. The compound of claim 2, wherein R₂, R₃ and R₄ is each selected from the group consisting of hydrogen, hydroxy, nitro, C₁₋₈alkoxy, and optionally substituted amino, wherein amino is optionally substituted with C₁₋₈acyl.

12. The compound of claim 2, wherein R₅ is one substituent selected from the group consisting of hydrogen, halogen, C₁₋₈alkyl and C₁₋₈alkyl-hydroxy.

13. A compound of formula (Ib): 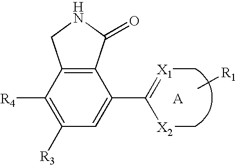
or a form thereof, wherein
the A(X₁,X₂) ring system moiety is selected from the group consisting of pyrrol-2-yl, indol-2-yl and benzimidazol-2-yl;
R₁ is one substituent selected from the group consisting of hydrogen, hydroxy and optionally substituted C₁₋₁₈alkoxy and carbonyl-C₁₋₈alkoxy, wherein C₁₋₈alkoxy is optionally substituted with one substituent selected from the group consisting of hydroxy, aryl-R₅, and heterocyclyl-R₅;
R₃ is selected from the group consisting of hydrogen, hydroxy, and C₁₋₈alkoxy;
R₄ is selected from the group consisting of hydrogen, nitro and optionally substituted amino, wherein amino is optionally substituted with C_{1 -8}acyl; and
R₅ is one substituent selected from the group consisting of hydrogen, C₁₋₈alkyl and C₁₋₈alkyl-hydroxy.

14. A compound selected from the group consisting of:
7-{5-[3-(4-ethyl-piperazin-1-yl)-propoxy]-1H-indol-2-yl}-2,3-dihydro-isoindol-1-one,
7-{5-[3-(4-hydroxymethyl-piperidin-1-yl)-propoxy]-1H-indol-2-yl}-2,3-dihydro-isoindol-1-one,
7-(5-methoxy-1H-indol-2-yl)-2,3-dihydro-isoindol-1-one,
7-(5-hydroxy-1H-indol-2-yl)-2,3-dihydro-isoindol-1-one,
7-[5-(3-hydroxy-propoxy)-1H-indol-2-yl]-2,3-dihydro-isoindol-1-one,
5-methoxy-7-(5-methoxy-1H-indol-2-yl)-2,3-dihydro-isoindol-1-one,
7-(1H-indol-2-yl)-2,3-dihydro-isoindol-1-one,
7-[5-(2-morpholin-4-yl-ethoxy)-1H-indol-2-yl]-2,3-dihydro-isoindol-1-one,
7-[5-(3-piperidin-1-yl-propoxy)-1H-indol-2-yl]-2,3-dihydro-isoindol-1-one,
7-[5-(2-piperidin-1-yl-ethoxy)-1H-indol-2-yl]-2,3-dihydro-isoindol-1-one,
7-(5-methoxy-1H-benzoimidazol-2-yl)-2,3-dihydro-isoindol-1-one,
7-(1H-pyrrol-2-yl)-2,3-dihydro-isoindol-1-one,
5-benzyloxy-2-(6-hydroxy-3-oxo-2,3-dihydro-1H-isoindol-4-yl)-indole-1-carboxylic acid tert-butyl ester,
7-(5-benzyloxy-1H-benzoimidazol-2-yl)-6-hydroxy-2,3-dihydro-isoindol-1-one,
5-methoxy-2-(7-nitro-3-oxo-2,3-dihydro-1H-isoindol-4-yl)-indole-1-carboxylic acid tert-butyl ester,
7-(5-methoxy-1H-indol-2-yl)-4-nitro-2,3-dihydro-isoindol-1-one,
4-amino-7-(5-methoxy-1H-indol-2-yl)-2,3-dihydro-isoindol-1-one,
N-[7-(5-methoxy-1H-indol-2-yl)-1-oxo-2,3-dihydro-1H-isoindol-4-yl]-acetamide,
7-(5-benzyloxy-1H-indol-2-yl)-2,3-dihydro-isoindol-1-one,
7-(1H-Indol-3-yl)-2,3-dihydro-isoindol-1-one,
2-(1,3-dioxo-2,3-dihydro-1H-isoindol-4-yl)-5-methoxy-indole-1-carboxylic acid tert-butyl ester,
4-(5-methoxy-1H-indol-2-yl)-isoindole-1,3-dione,
2-(3-oxo-2,3-dihydro-1H-isoindol-4-yl)-pyrrole-1-carboxylic acid tert-butyl ester,
2-(2-methyl-3-oxo-2,3-dihydro-1H-isoindol-4-yl)-pyrrole-1-carboxylic acid tert-butyl ester,
2-methyl-7-(1H-pyrrol-2-yl)-2,3-dihydro-isoindol-1-one,
2-(3-oxo-2,3-dihydro-1H-isoindol-4-yl)-indole-1-carboxylic acid tert-butyl ester,
5-methoxy-2-(3-oxo-2,3-dihydro-1H-isoindol-4-yl)-indole-1-carboxylic acid tert-butyl ester,
5-hydroxy-2-(3-oxo-2,3-dihydro-1H-isoindol-4-yl)-indole-1-carboxylic acid tert-butyl ester,
2-(3-oxo-2,3-dihydro-1H-isoindol-4-yl)-5-(3-piperidin-1-yl-ethoxy)-indole-1-carboxylic acid tert-butyl ester,
5-(2-morpholin-4-yl-ethoxy)-2-(3-oxo-2,3-dihydro-1H-isoindol-4-yl)-indole-1-carboxylic acid tert-butyl ester,
5-Methoxy-2-(6-methoxy-3-oxo-2,3-dihydro-1H-isoindol-4-yl)-indole-1-carboxylic acid tert-butyl ester,
5-benzyloxy-2-(3-oxo-2,3-dihydro-1H-isoindol-4-yl)-indole-1-carboxylic acid tert-butyl ester, and
5-benzyloxy-2-(7-nitro-3-oxo-2,3-dihydro-1H-isoindol-4-yl)-indole-1-carboxylic acid tert-butyl ester.

15. The compound of any of claim 1 to 14, wherein the compound is an isolated form thereof.

16. The compound of claim 1, wherein the compound or a form thereof is an inhibitor of ATP-protein kinase interactions.

17. The compound of claim 16, wherein the protein kinase is selected from a tyrosine kinase.

18. The compound of claim 16, wherein the protein kinase is selected from VEGF-R2 or Aurora-A.

19. A pharmaceutical composition comprising an effective amount of a compound of any of claim 1 to 15 and a pharmaceutically acceptable carrier.

20. The pharmaceutical composition of claim 19, wherein the effective amount of the compound is in a range of from about 0.001 mg/kg to about 300 mg/kg of body weight per day.

21. A process for preparing a pharmaceutical composition comprising the step of admixing a compound of any of claim 1 to 15 and a pharmaceutically acceptable carrier.

22. A method for treating or ameliorating a kinase mediated disorder in a subject in need thereof comprising administering to the subject an effective amount of a compound of any of claim 1 to 14.

23. The method of claim 22, wherein the kinase mediated disorder is an acute or chronic cancer selected from glioma cancers, epidermoid cancers, head and neck cancers, lung cancers, breast cancers, colorectal cancers, prostate cancers, gastric cancers, esophageal cancers, papillocarcinomas, Kaposi's sarcoma, leukemias and lymphomas; and associated pathologies is selected from abnormal cell proliferation, unregulated cell proliferation, tumor growth or tumor vascularization and associated pathologies selected from metastatic cancer cell invasion and migration, angiopathy, angiogenesis or chemotherapy-induced alopecia.

24. The method of claim 22, wherein the disorder is selected from acute inflammation, chronic inflammation, osteoarthritis, synovial pannus invasion in arthritis, multiple sclerosis, myasthenia gravis, diabetes mellitus, diabetic angiopathy, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin diseases or disorders, papilloma formation, psoriasis, dermatitis, eczema, seborrhea, central nervous system diseases, Alzheimer's disease, Parkinson's disease, depression, heart disease, hemangioma atheroma, mycotic infection, occular diseases, macular degeneration, diseases of the cornea, glaucoma, autoimmune disease, viral infections, cytomegalovirus, atherosclerosis, transplantation-induced vasculopathies, neointima formation, allergic-asthma, lung fibrosis, pulmonary fibrosis, chronic obstructive pulmonary disorder, acute, subacute or chronic forms of glomerulonephritis, glomerulosclerosis, congenital multicystic renal dysplasia, kidney fibrosis, diabetic retinopathy, rheumatoid arthritis or arterial restenosis.

25. The method of any of claim 22, wherein the kinase mediated disorder is selected from mycotic infection, cancer, tumor growth, tumor vascularization, angiopathy, angiogenesis, chemotherapy-induced alopecia or restenosis.

26. The method of claim 22, wherein the effective amount of the compound is from about 0.001 mg/kg/day to about 300 mg/kg/day.

27. The method of claim 22, further comprising administering the compound as an adjunct to chemotherapy and radiation therapy.

28. The method of claim 22, further comprising administering to the subject an effective amount of a combination product comprising at least one other therapeutic agent in combination with the compound.

29. A process for preparing a compound of any of claim 1 to 15 comprising the steps of: 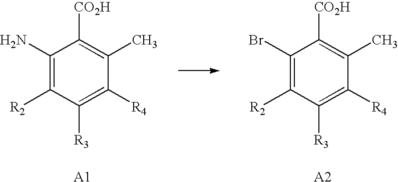
(a) reacting a Compound A1 with a suitable reagent to provide a brominated Compound A2; 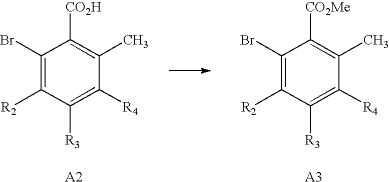
(b) reacting Compound A2 with an alkylating agent to provide a Compound A3; 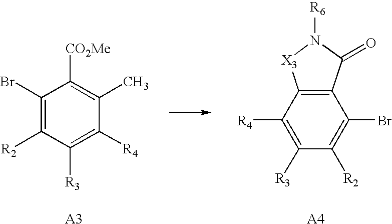
(c) reacting Compound A3 with a solution of a suitable reagent or a mixture thereof in the presence of a base to provide a Compound A4; and 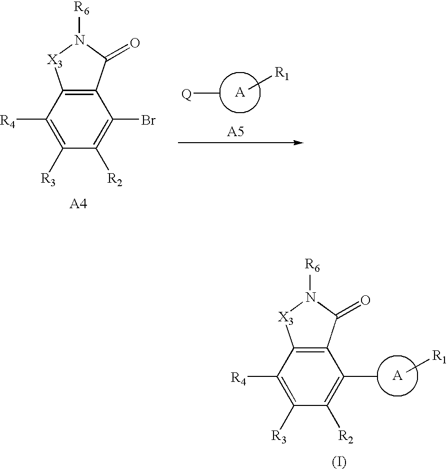
(d) reacting Compound A4 with a Compound A5 (wherein Q is a boronic acid or ester and the like) in the presence of a palladium catalyst to provide a compound of formula (I); or,
(e) reacting Compound A4 with a boronating reagent to form a boronated intermediate amenable for further reaction with a bromine substituted Compound A5 (wherein Q is bromine and the like) to thus provide a compound of formula (I).

30. A process for preparing a compound of any of claims 1 to 15 comprising the steps of: 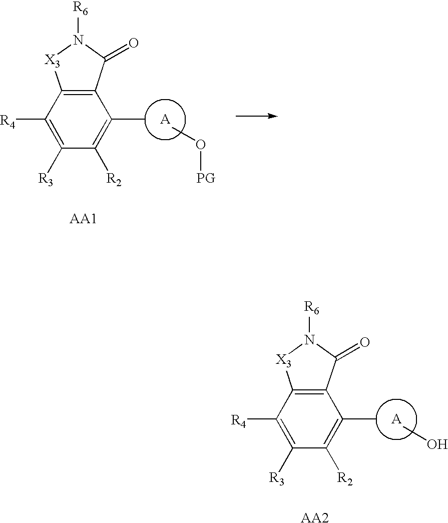
(a) reacting Compound AA1 with a suitable deprotecting reagent to provide a Compound AA2; and 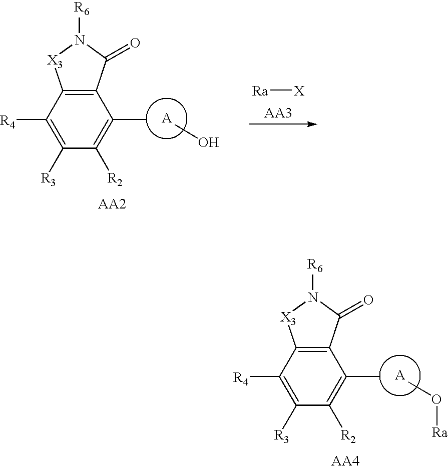
(b) reacting Compound AA2 with a Compound AA3 and suitable a base to provide a Compound AA4.

31. A process for preparing a compound of any of claims 1 to 15 comprising the step of: 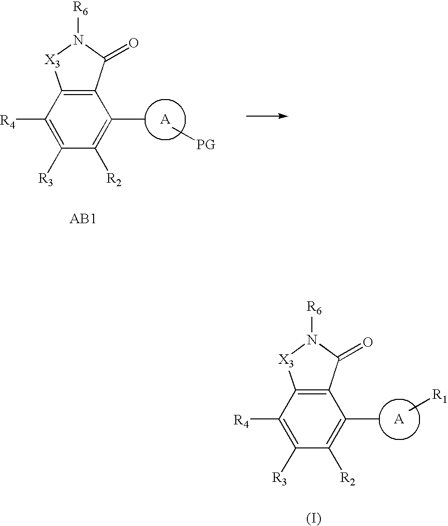
(a) reacting Compound AB1 with a suitable deprotecting reagent to provide a compound of formula (I).

32. A process for preparing a compound of any of claims 1 to 15 comprising the steps of: 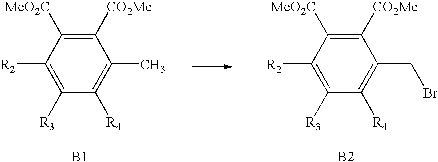
(a) reacting Compound B1 with a brominating reagent to provide a Compound B2; 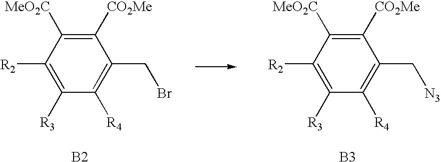
(b) reacting Compound B2 is reacted with an azide salt to provide a Compound B3; 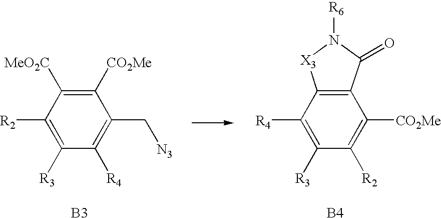
(c) reacting Compound B3 with a reducing agent to provide a cyclized Compound B4; 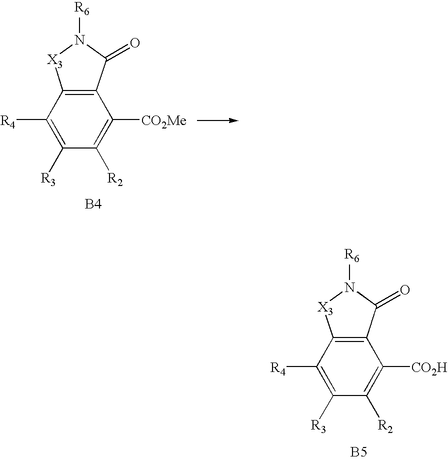
(d) reacting Compound B4 with a suitable base to afford a Compound B5; and 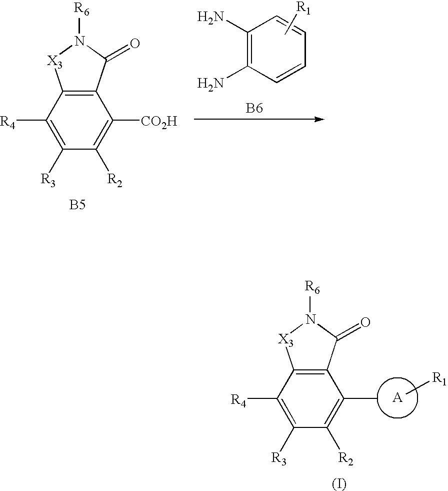
(e) reacting Compound B5 with a Compound B6 provide a compound of formula (I).

33. A process for preparing a compound of any of claims 1 to 15 comprising the steps of: 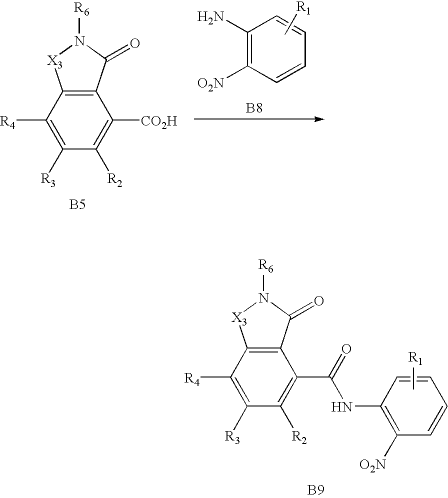
(a) reacting Compound B5 with a Compound B8 to provide a Compound B9; and 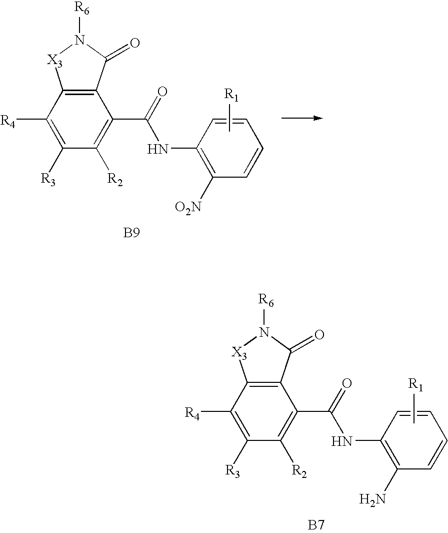
(b) reacting Compound B9 to provide a Compound B7; or 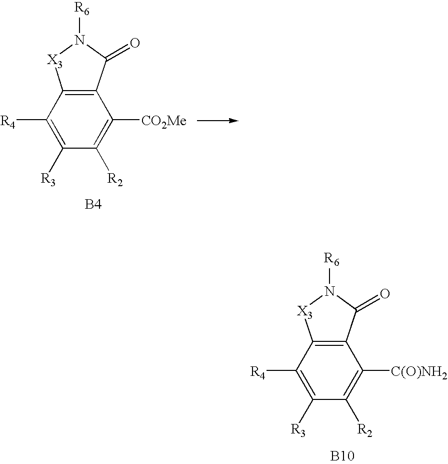
(c) reacting Compound B4 provide a Compound B10; and 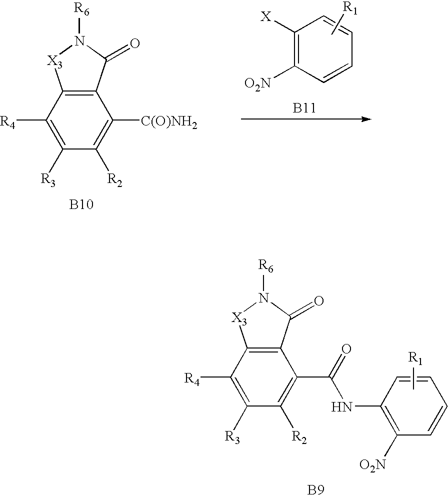
(d) reacting Compound B10 with a Compound B11 to provide a Compound B9; or 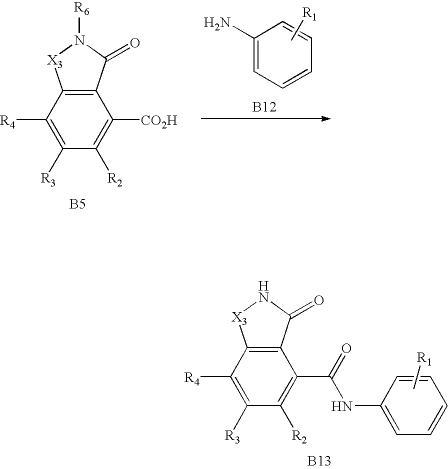
(e) reacting a Compound B5 with a Compound B12 to provide a Compound B13; and 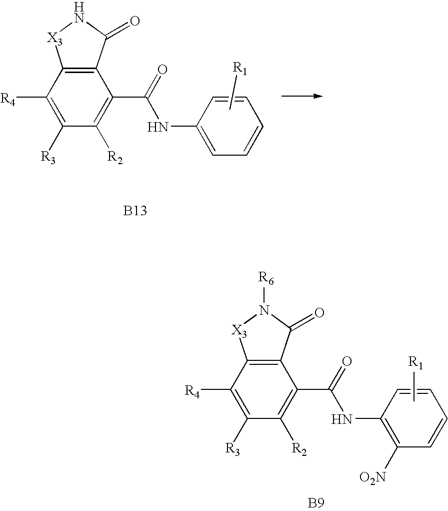
(f) reacting Compound B13 to provide a Compound B9; or 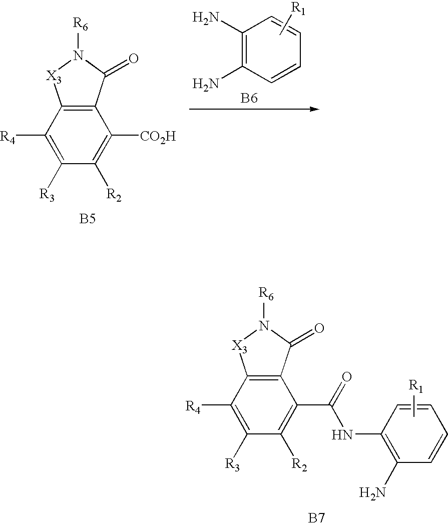
(g) reacting Compound B5 with a Compound B6 to provide a Compound 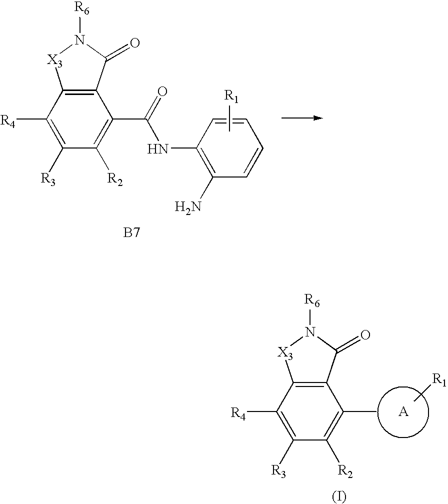
(h) reacting Compound B7 to provide a compound of formula (I).

34. A process for preparing a compound of any of claims 1 to 15 comprising the steps of: 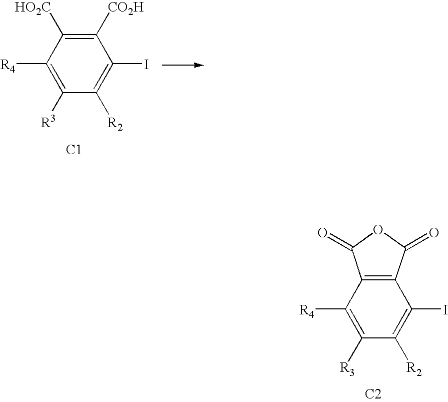
(a) reacting Compound C1 to provide a Compound C2; and 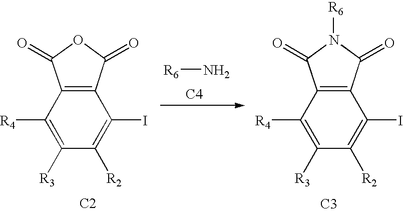
(b) reacting Compound C2 with a Compound C4 to provide a Compound C3; and 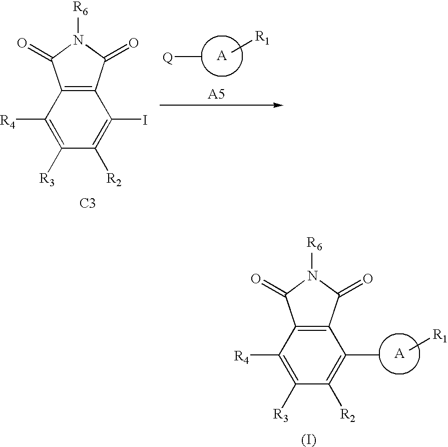
(c) reacting a Compound C3 with a Compound A5 to provide compound of formula (I); or
(d) reacting a Compound C3 with a boronating reagent to form a boronated intermediate that is further reacted with a bromine substituted Compound A5 to provide a compound of formula (I).

35. A process for preparing a compound of any of claims 1 to 15 comprising the steps of: 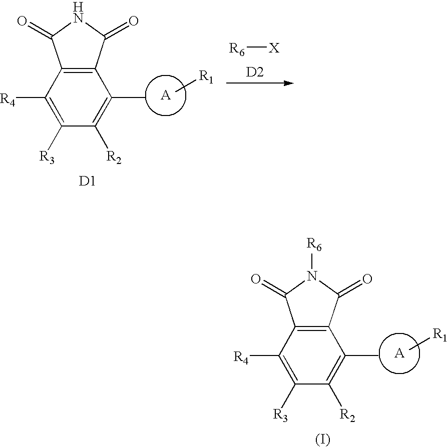
(a) reacting Compound D1 with a Compound D2 to provide a compound of formula (I) wherein X₃ is —C(O)—; or
(b) treating the compound of formula (I), wherein X₃ is —C(O)—, with a reducing agent to provide a compound of the formula (I), wherein X₃ is CH₂.
